(19) United States
(12) Patent Application Publication
(10) Pub. No.: US 2025/0333780 A1
(43) Pub. Date: Oct. 30, 2025

(54) METHOD OF DETECTING SIGNATURES OF GENETIC INSTABILITY

(71) Applicant: LUCENCE LIFE SCIENCES PTE. LTD., Singapore (SG)

(72) Inventors: Jonathan POH, Singapore (SG); Yukti CHOUDHURY, Singapore (SG); Min-Han TAN, Singapore (SG)

(21) Appl. No.: 18/868,513

(22) PCT Filed: May 24, 2023
(86) PCT No.: PCT/SG2023/050363

§ 371 Date: Nov. 22, 2024

(30) Foreign Application Priority Data
May 25, 2022 (SG) 10202205703V
Dec. 2, 2022 (SG) 10202260305W

(51) Int. Cl. C12Q 1/6858; C12Q 1/6883

(52) U.S. Cl. CPC C12Q 1/6858; C12Q 1/6883; C12Q 2600/156

(57) ABSTRACT

Disclosed is a method of detecting signatures of genetic instability within a nucleic acid sample, comprising: (a) identifying a plurality of single nucleotide polymorphism (SNPs) at one or more pre-determined intervals across (i) one or more target chromosome arms and/or (ii) one or more target genes; (b) performing a plurality of multiplexed PCR reactions using a plurality of forward and reverse primer pairs that are capable of capturing the plurality of SNPs, wherein each primer comprises a target-specific sequence, a barcode sequence, and an adapter-specific sequence, thereby generating a plurality of amplicons; and (c) sequencing and analysing the plurality of amplicons. In particular, the signature of genetic instability is loss of heterozygosity (LOH). Also disclosed is a method of predicting and/or monitoring the response of a subject having a disorder associated with signatures of genetic stability towards treatment. In particular, the disorder is Homologous Recombination Deficiency (HRD).

### CROSS-REFERENCES TO RELATED APPLICATIONS

This application is a national-stage application under 35 U.S.C. § 371 of International Application No. PCT/SG2023/050363, filed May 24, 2023, which claims the benefit of priority of Singapore Provisional Application No. 10202205703V, filed May 25, 2022, and Singapore Provisional Application No. 10202260305W, filed Dec. 2, 2022, the contents of which are being hereby incorporated by reference in their entirety for all purposes.

### SEQUENCE LISTING

A computer-readable form (CRF) sequence listing having file name 80397PCT_SQL.xml (11,848 bytes), created on May 19, 2023, is incorporated herein by reference. The nucleic and amino acid sequences listed in the accompanying sequence listing are shown using standard abbreviations as defined in 37 C.F.R. § 1.822.

### FIELD OF INVENTION

The present disclosure generally relates to a method of detecting signatures of genetic instability. In particular, the present invention relates to a method of detecting signatures of genetic instability using nucleic acid.

### BACKGROUND

DNA repair mechanisms play a role in maintaining the integrity of the human genome and to prevent cancer. The major DNA repair mechanisms in human include homologous recombination repair, non-homologous end joining repair, DNA mismatch repair, base excision repair, and nucleotide excision repair mechanisms. A defect in any one of these mechanisms may lead to the manifestation of one or more types of genomic instability.

Homologous recombination deficiency (HRD) (i.e., a defect in the homologous recombination repair mechanism) is a defining molecular feature of several cancer types, including ovarian, prostate, and breast cancers, and is characterised by genetic alterations in BRCA1/2 and other homologous recombination repair (HRR) genes. Deficiency in homologous recombination repair results in genome-wide genomic instability, manifesting as loss of heterozygosity (LOH), large-scale state transitions (LST), or telomeric allelic imbalance (TAI), biomarkers that can be used to predict HRD. Patients with HRD-positive tumours derive clinical benefit from, for example, PARP inhibitor treatment, highlighting the need to accurately and sensitively identify such patients.

Conventional HRD testing is performed by Next-Generation Sequencing (NGS) in formalin fixed, paraffin embedded tumour tissue DNA and involves either the detection of mutations in key HRR genes, signatures of genomic instability (including LOH, TAI, and LST), or a combination of the two. Detection of genomic instability signatures identifies additional patients who may benefit from, for example, PARP inhibitor therapy. However, the conventional method comes with high risks, cost, and complications associated with tissue biopsy. For example, conventional HRD tests generally require quantities of DNA≥30 ng and broad genome coverage which may not be amenable to testing in, for example, plasma cell-free DNA (cfDNA). Liquid biopsy from cfDNA provides an alternative avenue for the swift, accurate, and non-invasive molecular characterisation of tumours. Measurement of plasma cfDNA for the purposes of molecular characterisation of tumours possesses several clear advantages over tissue-based testing. Tissue-based testing is invasive and comes with risks and complications due to the inherent hard-to-access nature of many tumour lesions. Conversely, plasma-based liquid biopsy requires only a single draw of blood, enabling non-invasive serial monitoring of disease progression. Liquid biopsy also enables a quicker turnaround time, allowing faster treatment decisions to be reached, positioning it as an attractive alternative to tissue-based testing. In addition, such method can be used to probe the presence of circulating tumour DNA (ctDNA) found within cfDNA.

Although liquid biopsy-based detection methods for HRD exist, present options are limited to the detection of genetic mutations in HRR genes, missing a significant subset of patients that possess genomic instability without genetic mutations in key HRR genes, who may similarly benefit from treatment such as PARP inhibitor treatment. Such an approach (which only detects genetic mutations in HRR genes) severely limits the utility of liquid biopsy in HRD detection, as HRD-positive, HRR gene alteration-negative patients represent a significant population which also benefit from, for example, PARP inhibitor therapy as mentioned above. Additionally, in patients possessing germline BRCA1/2 deleterious mutations, loss of the wild-type allele (LOH) is a key aspect of tumourigenesis, and has been highlighted as a potential predictor of therapy response, establishing the need to demonstrate gene-level LOH in addition to identifying genetic mutations within key HRR genes for the identification of HRD-positive patients.

There are three main challenges posed by using cfDNA as an analyte. First, the fraction of ctDNA in cfDNA is often low, and requires highly sensitive methods of DNA detection and enumeration. In contrast, tissue samples are often enriched with tumour DNA, and contamination with non-tumour DNA often does not exceed 30%. Second, the concentration of cfDNA obtained from plasma can be low, particularly in patients with early-stage disease. Hence, while tissue-based testing can partially circumvent the need for high sensitivity methods by using higher quantities of input DNA, such an approach is impractical in liquid biopsy. Finally, for the detection of global LOH, the low sensitivity in tissue-based methods can be compensated for by having broad genomic coverage. Dependence on a large number of single nucleotide polymorphisms (SNPs) (typically genome-wide coverage) is required to provide sufficient resolution, for example, for global LOH detection. Existing analysis methods used for global LOH determination depend on broad genomic coverage, and include 1) enumeration of the number of LOH events exceeding 15 Mb in length, 2) determination of the fraction of length of continuous LOH sites compared to the length of all informative polymorphic sites measured, and 3) determination of the fraction of number of LOH sites compared to the number of all informative polymorphic sites measured. In cfDNA-based approaches, high sensitivity is typically achieved by ultradeep sequencing, which is highly cost-inefficient when coupled with broad genomic coverage, and does not lend well to implementation in routine clinical practice.

In addition to the limitations posed by cfDNA as analyte, a mutation-based HRD detection approach is incomplete. Knudson's two-hit model hypothesises that the inactivation of both alleles of tumour suppressor genes such as BRCA1/2 is required for tumourigenesis. In both breast and ovarian cancer, the most common mechanism whereby the second allele is lost following a deleterious BRCA1/2 mutation is through LOH. Hence, detection of mutations in HRR genes alone is insufficient for the comprehensive identification of HRD-positive patients.

Thus, there is a need to provide a method for the detection of one or more signatures of genetic instability (such as LOH, LST and TAI) that overcomes at least one or more of the disadvantages described above. There is also a need to provide a method for the detection of one or more signatures of genetic instability at chromosome-level, gene-level and/or global level using nucleic acid (such as cfDNA and tissue DNA) that is cost effective and highly sensitive.

### SUMMARY

In a first aspect, the present disclosure refers to a method of detecting the presence or absence of one or more signatures of genetic instability at chromosome-level and/or gene-level within a nucleic acid sample, comprising the steps of:
(a) identifying a plurality of single nucleotide polymorphism (SNPs) at one or more pre-determined intervals across:
   (I) one or more target chromosome arms, wherein each target chromosome arm comprises a plurality of genes; and/or
   (II) one or more target genes;
(b) performing a plurality of multiplexed PCR reactions using:
   (I) a plurality of forward and reverse primer pairs that are capable of capturing the plurality of SNPs identified across the one or more target chromosome arms in step (a)(I),
      wherein each primer of the plurality of forward and reverse primer pairs comprises a target-specific sequence capable of capturing at least one SNP in the plurality of the SNPs identified across the one or more target chromosome arms in step (a)(I),
      wherein each forward primer and/or reverse primer of the plurality of forward and reverse primer pairs comprise(s) a barcode sequence on the 5′ end of the target-specific sequence,
      wherein each primer of the plurality of forward and reverse primer pairs comprises an adapter-specific sequence; and/or
      (II) a plurality of forward and reverse primer pairs that are capable of capturing the plurality of SNPs identified across the one or more target genes in step (a)(II),
      wherein each primer of the plurality of forward and reverse primer pairs comprises a target-specific sequence capable of capturing at least one SNP in the plurality of the SNPs identified across the one or more target genes in step (a)(II),
      wherein each forward primer and/or reverse primer of the plurality of forward and reverse primer pairs comprise(s) a barcode sequence on the 5′ end of the target-specific sequence,
      wherein each primer of the plurality of forward and reverse primer pairs comprises an adapter-specific sequence, thereby generating a plurality of amplicons;
(c) using the plurality of amplicons from step (b) to generate a plurality of sequencing reads with a next-generation sequencing platform;
(d) deriving a consensus sequence read of each sequence from the plurality of sequencing reads obtained from step (c);
(e) performing a sequence alignment of the consensus sequence reads obtained from step (d) to a reference genome;
(f) performing variant calling based on the sequence alignment obtained from step (e) to calculate variant allele frequency (VAF);
(g) determining and enumerating a plurality of informative polymorphic sites from the VAF obtained in step (f), wherein an informative polymorphic site is defined as a site comprising between 5% and 95% VAF;
(h) calculating the allelic ratio (AR) at each informative polymorphic site of the plurality of informative polymorphic sites determined in step (g), wherein AR is defined as a ratio of a major allele A to a minor allele B, wherein
   (I) if the AR at an informative polymorphic site is equal to or higher than a pre-determined threshold value, said informative polymorphic site is classified as “genetically unstable”; and
   (II) if the AR at an informative polymorphic site is lower than a pre-determined threshold value, said informative polymorphic site is classified as “genetically stable” (not genetically unstable); and
   wherein if the one or more target chromosome arms and/or the one or more target genes are determined to be “positive”, then one or more signatures of genomic instability are determined to be present at chromosome-level and/or gene-level within the nucleic acid sample, and
   wherein if there is/are no target chromosome arm and/or target gene that is/are determined to be “positive”, then one or more signatures of genomic instability are determined to be absent at chromosome-level and/or gene-level within the nucleic acid sample;
   thereby detecting the presence or absence of one or more signatures of genomic instability at chromosome-level and/or gene-level within the nucleic acid sample based on the results obtained in step (i).

In a second aspect, the present disclosure refers to a kit for detecting the presence or absence of one or more signatures of genetic instability at chromosome-level and/or gene-level within a nucleic acid sample according to the method disclosed herein, wherein the kit comprises:
a plurality of forward and reverse primer pairs that are capable of capturing a plurality of SNPs identified across one or more target chromosome arms as defined in the first aspect; and/or
a plurality of forward and reverse primer pairs that are capable of capturing a plurality of SNPs identified across one or more target genes as defined in the first aspect.

In a third aspect, the present disclosure refers to a method of predicting and/or monitoring the response of a subject having a disorder associated with one or more signatures of genetic instability towards treatment with one or more poly (ADP-ribose) polymerase inhibitors, comprising detecting the presence or absence of one or more signatures of genetic instability at chromosome-level and/or gene level according to the method disclosed herein.

### BRIEF DESCRIPTION OF DRAWINGS

The invention will be better understood with reference to the detailed description when considered in conjunction with the non-limiting examples and the accompanying drawings, in which:

FIG. 1 (comprised of FIGS. 1A, 1B and 1C) shows the panel-wide distribution of single nucleotide polymorphisms (SNPs). FIG. 1A shows an overview of SNP placement in chromosome 14. FIG. 1B shows the sparse uniform chromosome-level SNPs for broad chromosome arm coverage. FIG. 1C shows the dense gene-level SNP coverage for determination of gene-specific loss of heterozygosity.

FIG. 2 (comprised of FIGS. 2A and 2B) shows the method of detection for loss of heterozygosity (LOH). FIG. 2A shows that SNPs are captured in amplicons using forward and reverse primers (represented by (→) and (←) respectively) designed to incorporate molecular barcodes and partial sequencing adapters. Amplicons are completed for next-generation sequencing with a further round of PCR amplification to integrate full sequencing adapters. FIG. 1B shows LOH detection based on SNP allelic ratio. When no LOH is present (right bar), the proportions of A (major) and B (minor) alleles at a heterozygous SNP are equivalent. When LOH is present in a tumour sample (left and middle bars), an imbalance of the allelic ratio is observed. The magnitude of this ratio (A allele to B allele) is indicative of the tumour fraction within a given sample, where the sample is a mix of normal and tumour DNA.

FIG. 3 (comprised of FIGS. 3A and 3B) shows the accuracy and precision of the variant allele frequencies (VAFs) determined by the method of the present disclosure. FIG. 3A shows the range of variant allele frequencies (VAFs) of all variants detected between 10% and 90% VAF from sequencing 2.5 ng of 8 genomic DNA samples. FIG. 3B shows the distribution of standard deviation of VAF measurements across 693 heterozygous SNPs from sequencing 5-10 replicates of 5 cfDNA samples.

FIG. 4 (comprised of FIGS. 4A and 4B) shows that the method of the present disclosure can be used for evaluating the type of loss of heterozygosity (LOH), as disclosed in step (j) of the method of the first aspect. FIG. 4A shows that for copy number loss LOH, a deviation in allelic ratio (top panel) is coupled with a decrease in copy number (copy number loss) (bottom panel). FIG. 4B shows that for copy neutral LOH, only a deviation in allelic ratio is observed. Broken lines indicate the threshold for calling LOH (allelic ratio) and copy number change (copy number). The x-axis in all panels approximates chromosomal positions and copy number is calculated as a fold-change of sequencing coverage compared to the expected normal coverage from a set of baseline samples.

FIG. 5 shows a flowchart illustrating the data analysis workflow for identifying gene-specific loss of heterozygosity (LOH) and chromosome-level LOH as well as the presence of global LOH signature. Informative polymorphic sites are identified as disclosed in step (g) of the method of the first aspect. The informative polymorphic sites are in turn used to determine the presence of LOH at gene-level and chromosome level (steps (h) and (i)) as well as at global-level (steps (k) and (l)), which can then be used to determine the HRD status in a nucleic acid sample.

FIG. 6 (comprised of FIGS. 6A and 6B) shows that gene-specific LOH can be detected at low tumour fractions (TF) with accurate TF estimation. FIG. 6A shows an example of copy neutral LOH (cnLOH) and FIG. 6B shows an example of copy number loss LOH (CNL-LOH) detection. Tumour fractions were generated by admixing (A) HCC1937 DNA with normal HCC1937BL DNA or (B) HCC1395 DNA with normal HCC1395BL DNA. Hit and missed calls are indicated by the symbols “X” and “O” respectively.

FIG. 7 shows that the global loss of heterozygosity (LOH) signature can be detected at low tumour fractions (TF). Tumour fractions were generated in silico by admixing two cfDNA samples with known HRD-positive status with their respective buffy coat gDNA.

### DETAILED DESCRIPTION

The present disclosure describes a method of detecting one or more signatures of genetic instability, such as loss of heterozygosity (LOH), large-scale transitions (LST), and telomeric allelic imbalance (TAI), within a nucleic acid sample. The present disclosure solves the unmet need of identifying (A) signatures of genomic instability and (B) gene-specific signatures of genetic instability (such as LOH in key HRR genes in cfDNA), both of which are essential components of comprehensive detection of DNA repair deficiency disorder, such as HRD detection. In the present disclosure, the use of cfDNA as an analyte for the detection of HRD-related signatures of genetic instability (such as LOH) is also made possible through the design of a multiplex amplicon-based NGS assay encompassing SNP loci across the genome and within key HRR genes.

In a first aspect, the present disclosure refers to a method of detecting the presence or absence of one or more signatures of genetic instability at chromosome-level and/or gene-level within a nucleic acid sample, comprising the steps of:
(a) identifying a plurality of single nucleotide polymorphism (SNPs) at one or more pre-determined intervals across:
   (I) one or more target chromosome arms, wherein each target chromosome arm comprises a plurality of genes; and/or
   (II) one or more target genes;
(b) performing a plurality of multiplexed PCR reactions using:
   (I) a plurality of forward and reverse primer pairs that are capable of capturing the plurality of SNPs identified across one or more target chromosome arms in step (a)(I),
      wherein each primer of the plurality of forward and reverse primer pairs comprises a target-specific sequence capable of capturing at least one SNP in the plurality of the SNPs identified across one or more target chromosome arms in step (a)(I),
      wherein each forward primer and/or reverse primer of the plurality of forward and reverse primer pairs comprise(s) a barcode sequence on the 5′ end of the target-specific sequence,
      wherein each primer of the plurality of forward and reverse primer pairs comprises an adapter-specific sequence; and/or
   (II) a plurality of forward and reverse primer pairs that are capable of capturing the plurality of SNPs identified across the one or more target genes in step (a)(II),
      wherein each primer of the plurality of forward and reverse primer pairs comprises a target-specific sequence capable of capturing at least one SNP in the plurality of the SNPs identified across the one or more target genes in step (a)(II),
      wherein each forward primer and/or reverse primer of the plurality of forward and reverse primer pairs comprise(s) a barcode sequence on the 5′ end of the target-specific sequence,
      wherein each primer of the plurality of forward and reverse primer pairs comprises an adapter-specific sequence, thereby generating a plurality of amplicons;
(c) using the plurality of amplicons from step (b) to generate a plurality of sequencing reads with a next-generation sequencing platform;
(d) deriving a consensus sequence read of each sequence from the plurality of sequencing reads obtained from step (c);
(e) performing a sequence alignment of the consensus sequence reads obtained from step (d) to a reference genome;
(f) performing variant calling based on the sequence alignment obtained from step (e) to calculate variant allele frequency (VAF);
(g) determining and enumerating a plurality of informative polymorphic sites from the VAF obtained in step (f), wherein an informative polymorphic site is defined as a site comprising between 5% and 95% VAF;
(h) calculating the allelic ratio (AR) at each informative polymorphic site of the plurality of informative polymorphic sites determined in step (g), wherein AR is defined as a ratio of a major allele A to a minor allele B, wherein
   (I) if the AR at an informative polymorphic site is equal to or higher than a pre-determined threshold value, said informative polymorphic site is classified as “genetically unstable”; and
   (II) if the AR at an informative polymorphic site is lower than a pre-determined threshold value, said informative polymorphic site is classified as “genetically stable” (not genetically unstable); and
(i) determining whether the one or more target chromosome arms and/or the one or more target genes are “positive” for one or more signatures of genetic instability, wherein
   (I) if a target chromosome arm comprises a minimum pre-determined number of informative polymorphic sites obtained from step (g) and if at least 50% of the informative polymorphic sites are classified as “genetically unstable” in step (h)(I), said target chromosome arm is determined to be “positive” for one or more signatures of genetic instability at chromosome-level; and/or
   (II) if a target gene comprises a minimum pre-determined number of informative polymorphic sites obtained from step (g) and if at least 30% of the informative polymorphic sites are classified as “genetically unstable” in step (h)(I), said target gene is determined to be “positive” for one or more signatures of genetic instability at gene-level;
   wherein if the one or more target chromosome arms and/or the one or more target genes are determined to be “positive”, then one or more signatures of genomic instability are determined to be present at chromosome-level and/or gene-level within the nucleic acid sample, and
   wherein if there is/are no target chromosome arm and/or target gene that is/are determined to be “positive”, then one or more signatures of genomic instability are determined to be absent at chromosome-level and/or gene-level within the nucleic acid sample;
thereby detecting the presence or absence of one or more signatures of genomic instability at chromosome-level and/or gene-level within the nucleic acid sample based on the results obtained in step (i).

The term “signature of genetic instability” refers to the resulting effect, feature, or manifestation of a disease or condition that causes genetic instability. In one example, the disease or condition may be caused by somatic and/or germline mutation. The signature of genetic instability may refer to any signature that is known in the art, such as loss of heterozygosity (LOH), large-scale state transitions (LST), and telomeric allelic imbalance (TAI). In one example, the signature of genetic instability is LOH. LOH refers to a type of allelic imbalance where a heterozygous locus within the nucleic acid becomes homozygous or hemizygous due to the loss of one parental allele. LST refers to the occurrence of chromosomal breakage of 10 megabases (Mb) or more between two regions within the nucleic acid. TAI refers to a type of allelic imbalance occurring from a given position to the sub-telomere of a chromosome, but without crossing the centromere of the chromosome. In one example, the signature of genetic instability is the resulting effect, feature, or manifestation of a defective DNA repair pathway or a DNA repair deficiency disorder. The DNA repair deficiency disorder may include, but is not limited to Homologous Recombination Deficiency (HRD), Non-Homologous End-Joining (NHEJ) Deficiency, DNA mismatch repair (MMR) deficiency, nucleotide excision repair (NER) deficiency, and base excision repair (BER) deficiency. In one example, the DNA repair deficiency disorder is HRD.

In one example, the disclosed method is used to detect the presence or absence of one or more signatures of genetic instability at chromosome-level within a nucleic acid sample. In one example, the disclosed method is used to detect the presence or absence of one or more signatures of genetic instability at gene-level within a nucleic acid sample. In one example, the disclosed method is used to simultaneously detect the presence or absence of one or more signatures of genetic instability at chromosome-level and gene-level within a nucleic acid sample.

The term “single nucleotide polymorphism (SNP)” refers to variation in a single nucleotide at a specific genomic position or specific position in the genome, differing from the nucleotide defining the position in the reference genome. The reference genome may be obtainable from public databases. The variation in the single nucleotide may be due to substitution. The SNPs may be naturally occurring or inherited. In one example, the SNPs are naturally occurring. In one example, the SNPs are naturally occurring germline substitution mutations. In one example, the SNPs are naturally occurring and may be present in any genes and/or any chromosomes arms found in a nucleic acid sample of a subject, regardless of the number of chromosome arms present or of the genotype of the nucleic acid of a subject. In one example, the SNPs that are naturally occurring are selected or identified or determined or pre-determined by population genetic studies. In one example, the SNPs are described as homozygous SNPs if they are found in homozygous loci or positions in the nucleic acid. In one example, the SNPs are described as hemizygous if they are found in hemizygous loci or positions in the nucleic acid. In another example, the SNPs are described as heterozygous SNPs if they are found in heterozygous loci or positions in the nucleic acid. In one example, the method of the present disclosure involves identifying a plurality of homozygous SNPs, hemizygous SNPs and/or heterozygous SNPs. In another example, the method of the present disclosure involves identifying a plurality of heterozygous SNPs. As used herein, the term “single nucleotide polymorphism (SNP)” can be used interchangeably with “single nucleotide sequence variation” and “point mutation”. The identification of SNPs may be guided by several criteria. In one example, SNPs with low population frequencies (such as less than 40% for chromosome-level SNPs and less than 10% for gene-level SNPs) are excluded. In another example, insertion-deletion mutations are excluded. In yet another example, tandem repeats are excluded.

The term “interval” refers to the distance in terms of number of base pairs or number of nucleotides across a sequence on a gene or chromosome arm or chromosome. The interval may be described in single base pair or in tens, hundreds, kilo (kb, thousands), mega (Mb, millions), or giga (Gb, billions) base pairs. The method of the present disclosure involves first identifying a plurality of SNPs at one or more pre-determined intervals across one or more target chromosome arms as disclosed in step (a)(I) and/or one or more target genes as disclosed in step (a)(II) of the first aspect. In one example, the method of the present disclosure involves identifying a plurality of SNPs at one or more pre-determined intervals across one or more target chromosome arms as disclosed in step (a)(I) of the first aspect. In one example, the method of the present disclosure involves identifying a plurality of SNPs at one or more pre-determined intervals across one or more target genes as disclosed in step (a)(II) of the first aspect. In one example, the method of the present disclosure involves simultaneously identifying a plurality of SNPs at one or more pre-determined intervals across one or more target chromosome arms as disclosed in step (a)(I) and one or more target genes as disclosed in step (a)(II) of the first aspect. In one example, the term “identifying” in the step of identifying a plurality of SNPs at one or more pre-determined intervals across one or more target chromosome arms as disclosed in step (a)(I) and/or one or more target genes as disclosed in step (a)(II) of the first aspect may be used interchangeably with the term “selecting”. In one example, the term “pre-determined intervals” may be used interchangeably with the term “pre-selected intervals”. In one example, “plurality” means at least two. Therefore, in one example, the plurality of SNPs identified at one or more pre-determined intervals across one or more target chromosome arms and/or one or more genes comprise at least two SNPs. The identification of the SNPs at one or more pre-determined intervals provides for the distribution of the SNPs across a target gene, a target chromosome arm, a target chromosome, or the genome as a whole. In one example, the plurality of SNPs are “densely” distributed across the target gene, the target chromosome arm, the target chromosome, or the genome as a whole. In another example, the plurality of SNPs are “sparsely” distributed across the target gene, the target chromosome arm, the target chromosome, or the genome as a whole. In one example, the distinction between “dense” and “sparse” distribution can be interpreted as an interval in terms of kb vs an interval in terms of Mb, respectively. In one example, the terms “dense” and “sparse” distribution are used to describe the distribution of SNPs within genes (with the longest gene being 2.2 kb) and chromosomes (which range from 48 to 249 Mb in length). In one example, the plurality of SNPs are sparsely distributed across the target chromosome arm. In one example, the plurality of SNPs are densely distributed across the target gene.

In one example, the pre-determined interval may be described as a “uniform interval” which refer to a balanced coverage of any target gene, target chromosome arm, target chromosome, or the genome as a whole, and therefore provides a guidance for identification of the plurality of SNPs in step (a) of the first aspect. This would prevent, for example, having 90% of the plurality of SNPs located within 10% of the chromosome arm and the remaining 10% of the plurality of SNPs located within 90% of the chromosome arm only. There are several factors that can preclude specific genomic regions from being targeted, for instance, if the genomic regions are SNP poor, or if the SNPs are found in low complexity genomic regions. In one example, the determination of the one or more pre-determined intervals (or pre-selected intervals) depends on the length of the target chromosome arm and the number of SNPs targeted within that chromosome arm. For instance, on chr1q (124 Mb), a regular or uniform interval could be 12.4 Mb per SNP for 10 SNPs, 6.2 Mb per SNP for 20 SNPs, etc. In contrast, on chr20p (28 Mb), a regular interval could be 2.8 Mb per SNP for 10 SNPs, or 1.4 Mb per SNP for 20 SNPs. In one example, the determination of the one or more pre-determined intervals (or pre-selected intervals) depends on the length of the target gene and the number of SNPs targeted within that gene. In one example, the target gene has a length of 7 kb to 867 kb. In one example, based on a minimum of 3 SNPs and an example of a target gene length that range from 7 kb to 867 kb, a lower limit of 2 kb and upper limit of 300 kb may be appropriate. In one example, the target gene with a length that range from 7 kb to 867 kb is a DNA repair pathway gene. In one example, the DNA repair pathway gene is a homologous recombination repair (HRR) gene. In one example, the target gene with a length that range from 7 kb to 867 kb may be, but is not limited to, AT-rich interaction domain 1A (ARID1A), ATM serine/threonine kinase (ATM), ATR serine/threonine kinase (ATR), ATRX chromatin remodeler (ATRX), BRCA1 associated protein 1 (BAP1), BRCA1 associated RING domain 1 (BARD1), BLM RecQ like helicase (BLM), BRCA1 DNA repair associated (BRCA1), BRCA2 DNA repair associated (BRCA2), BRCA1 interacting helicase 1 (BRIP1), cyclin dependent kinase 12 (CDK12), Checkpoint kinase 1 (CHEK1), Checkpoint kinase 2 (CHEK2), EMSY transcriptional repressor, BRCA2 interacting (EMSY), FA complementation group A (FANCA), FA complementation group C (FANCC), FA complementation group D2 (FANCD2), FA complementation group E (FANCE), FA complementation group F (FANCF), FA complementation group G (FANCG), FA complementation group I (FANCI), FA complementation group L (FANCL), FA complementation group M (FANCM), MRE11 homolog, double strand break repair nuclease (MRE11), nibrin (NBN), Partner and localizer of BRCA2 (PALB2), Phosphatase and tensin homolog (PTEN), RAD50 double strand break repair protein (RAD50), RAD51 recombinase (RAD51), RAD51 paralog B (RAD51B), RAD51 paralog C (RAD51C), RAD51 paralog D (RAD51D), RAD52 homolog, DNA repair protein (RAD52), RAD54 like (RAD54L), Replication protein A1 (RPA1), or X-ray repair cross complementing 2 (XRCC2). In one example, the determination of the one or more pre-determined intervals (or pre-selected intervals) depends on the presence of SNP “desert” (i.e., regions in the genome where there are an abnormally low number of SNPs).

In one example, the one or more pre-determined intervals for the plurality of SNPs identified across the one or more target chromosome arms comprise 1 to 20 Mb. In one example, the one or more pre-determined intervals for the plurality of SNPs identified across the one or more target chromosome arms comprise 2 to 19 Mb, or 3 to 18 Mb, or 4 to 17 Mb, or 5 to 16 Mb, or 6 to 15 Mb, or 7 to 14 Mb, or 8 to 13 Mb, or 9 to 12 Mb, or 10 to 11 Mb. In one example, the one or more pre-determined intervals for the plurality of SNPs identified across the one or more target chromosome arms comprise any number of base pairs between 1 to 2 Mb, or 2 to 3 Mb, or 3 to 4 Mb, or 4 to 5 Mb, or 5 to 6 Mb, or 6 to 7 Mb, or 7 to 8 Mb, or 8 to 9 Mb, or 9 to 10 Mb, or 10 to 11 Mb, or 11 to 12 Mb, or 12 to 13 Mb, or 13 to 14 Mb, or 14 to 15 Mb, or 15 to 16 Mb, or 16 to 17 Mb, or 17 to 18 Mb, or 18 to 19 Mb, or 19 to 20 Mb. In one example, the one or more pre-determined intervals for the plurality of SNPs identified across the one or more target chromosome arms comprise 2 to 10 Mb. In one example, the one or more pre-determined intervals for the plurality of SNPs identified across the one or more target chromosome arms may be lower than 2 Mb and/or higher than 10 Mb. In one example, the one or more pre-determined intervals for the plurality of SNPs identified across the one or more target chromosome arms comprise about 1 Mb, or about 2 Mb, or about 3 Mb, or about 4 Mb, or about 5 Mb, or about 6 Mb, or about 7 Mb, or about 8 Mb, or about 9 Mb, or about 10 Mb, or about 11 Mb, or about 12 Mb, or about 13 Mb, or about 14 Mb, or about 15 Mb, or about 16 Mb, or about 17 Mb, or about 18 Mb, or about 19 Mb, or about 20 Mb.

In one example, the one or more pre-determined intervals for the plurality of SNPs identified across the one or more target genes comprise 2 to 300 kb. In one example, the one or more pre-determined intervals for the plurality of SNPs identified across the one or more target genes comprise 10 to 290 kb, or 20 to 280 kb, or 30 to 270 kb, or 40 to 260 kb, or 50 to 250 kb, or 60 to 240 kb, or 70 to 230 kb, or 80 to 220 kb, or 90 to 210 kb, or 100 to 200 kb, or 110 to 190 kb, or 120 to 180 kb, or 130 to 170 kb, or 140 to 160 kb. In one example, the one or more pre-determined intervals for the plurality of SNPs identified across the one or more target genes comprise about 2 kb, or about 10 kb, or about 20 kb, or about 30 kb, or about 40 kb, or about 50 kb, or about 60 kb, or about 70 kb, or about 80 kb, or about 90 kb, or about 100 kb, or about 110 kb, or about 120 kb, or about 130 kb, or about 140 kb, or about 150 kb, or about 160 kb, or about 170 kb, or about 180 kb, or about 190 kb, or about 200 kb, or about 210 kb, or about 220 kb, or about 230 kb, or about 240 kb, or about 250 kb, or about 260 kb, or about 270 kb, or about 280 kb, or about 290 kb, or about 300 kb.

The target gene may be selected from any genes that are known or present in the nucleic acid (such as cfDNA) of a subject. In one example, the target gene may be a DNA repair pathway gene. In one example, the DNA repair pathway gene is a homologous recombination repair (HRR) gene. In one example, the target gene may include, but is not limited to AT-rich interaction domain 1A (ARID1A), ATM serine/threonine kinase (ATM), ATR serine/threonine kinase (ATR), ATRX chromatin remodeler (ATRX), BRCA1 associated protein 1 (BAP1), BRCA1 associated RING domain 1 (BARD1), BLM RecQ like helicase (BLM), BRCA1 DNA repair associated (BRCA1), BRCA2 DNA repair associated (BRCA2), BRCA1 interacting helicase 1 (BRIP1), cyclin dependent kinase 12 (CDK12), Checkpoint kinase 1 (CHEK1), Checkpoint kinase 2 (CHEK2), EMSY transcriptional repressor, BRCA2 interacting (EMSY), FA complementation group A (FANCA), FA complementation group C (FANCC), FA complementation group D2 (FANCD2), FA complementation group E (FANCE), FA complementation group F (FANCF), FA complementation group G (FANCG), FA complementation group I (FANCI), FA complementation group L (FANCL), FA complementation group M (FANCM), MRE11 homolog, double strand break repair nuclease (MRE11), nibrin (NBN), Partner and localizer of BRCA2 (PALB2), Phosphatase and tensin homolog (PTEN), RAD50 double strand break repair protein (RAD50), RAD51 recombinase (RAD51), RAD51 paralog B (RAD51B), RAD51 paralog C (RAD51C), RAD51 paralog D (RAD51D), RAD52 homolog, DNA repair protein (RAD52), RAD54 like (RAD54L), Replication protein A1 (RPA1), or X-ray repair cross complementing 2 (XRCC2).

The target chromosome arm may be selected from any chromosome arms from any chromosomes found in a subject. The chromosome may be an autosomal chromosome or a sex chromosome. In one example, the chromosome is an autosomal chromosome. An autosomal chromosome refers to any chromosome that is not a sex chromosome. In one example, the target chromosome arm is selected from any autosomal chromosomes found in a subject. In one example, the subject is a human and the target chromosome arm is selected from any one of the 22 pairs of autosomal chromosomes found in the human. In one example, the subject is a human and the target chromosome is a sex chromosome X or a sex chromosome Y. In one example, the target chromosome arm comprises a plurality of genes. In one example, the plurality of genes within the target chromosome arm may include any genes that are known or present in the genome of a subject and consequently in the nucleic acid sample from the subject. The genes may be protein coding or non-protein coding genes. In one example, the plurality of genes within the target chromosome arm may include one or more of the target genes as disclosed herein. In one example, the plurality of genes within the target chromosome arm may include one or more housekeeping genes. In one example, the plurality of genes within the target chromosome arm may include one or more of the target genes as disclosed herein and one or more housekeeping genes. In one example, “housekeeping genes” refer to highlight conserved genes which are essential for maintaining cellular function. In one example, the housekeeping genes may include, but are not limited to, Glucose-6-phosphate isomerase (GPI), FERM domain containing 8 (FRMD8), Small nuclear ribonucleoprotein D3 (SNRPD3), Proteasome subunit, beta type, 2 (PSMB2), TATA box binding protein (TBP), REL proto-oncogene, NF-kB subunit (REL), synaptosome associated protein 29 (SNAP29), Tubulin gamma complex associated protein 2 (TUBGCP2), Receptor accessory protein 5 (REEP5), Solute carrier family 4 member 1 adaptor protein (SLC4A1AP), Integrin subunit beta 7 (ITGB7), Protein-O-mannose kinase (POMK), ER membrane protein complex subunit 7 (EMC7), Nuclear autoantigenic sperm protein (NASP), Checkpoint with forkhead and ring finger domains (CHFR), Ribosomal RNA processing 1 (RRP1), Cytosolic iron-sulfur assembly component 1 (CIAO1), Pumilio RNA binding family member 1 (PUM1), Retention in endoplasmic reticulum sorting receptor 1 (RER1), Serine and arginine rich splicing factor 4 (SRSF4).

Following the identification of the plurality of SNPs across the one or more target chromosome arms and/or the one or more target genes, a plurality of multiplexed PCR reactions are performed by using a plurality of forward and reverse primer pairs designed to capture the plurality of SNPs identified, as disclosed in step (b) of the first aspect. In one example, the plurality of forward and reverse primer pairs that are capable of capturing the plurality of SNPs identified across the one or more target chromosome arms are designed as disclosed in step (b)(I):
wherein each primer of the plurality of forward and reverse primer pairs comprises a target-specific sequence capable of capturing at least one SNP in the plurality of the SNPs identified across the one or more target chromosome arms,
wherein each forward primer and/or reverse primer of the plurality of forward and reverse primer pairs comprise(s) a barcode sequence on the 5′ end of the target-specific sequence, and
wherein each primer of the plurality of forward and reverse primer pairs comprises an adapter-specific sequence.

In one example, the plurality of forward and reverse primer pairs that are capable of capturing the plurality of SNPs identified across the one or more target genes are designed as disclosed in step (b)(II):
wherein each primer of the plurality of forward and reverse primer pairs comprises a target-specific sequence capable of capturing at least one SNP in the plurality of the SNPs identified across the one or more target genes,
wherein each forward primer and/or reverse primer of the plurality of forward and reverse primer pairs comprise(s) a barcode sequence on the 5′ end of the target-specific sequence, and
wherein each primer of the plurality of forward and reverse primer pairs comprises an adapter-specific sequence.

In one example, the plurality of multiplexed PCR reactions are performed using a plurality of forward and reverse primer pairs that are capable of capturing the plurality of SNPs identified across the one or more target chromosome arms as disclosed in step (b)(I). In one example, the plurality of multiplexed PCR reactions are performed by using a plurality of forward and reverse primer pairs that are capable of capturing the plurality of SNPs identified across the one or more target genes as disclosed in step (b)(II). In one example, the plurality of multiplexed PCR reactions are performed by simultaneously using a plurality of forward and reverse primer pairs that are capable of capturing the plurality of SNPs identified across the one or more target chromosome arms as disclosed in step (b)(I) and a plurality of forward and reverse primer pairs that are capable of capturing the plurality of SNPs identified across one or more target genes as disclosed in step (b)(II).

In one example, each primer of the plurality of forward and reverse primer pairs disclosed in step (b)(I) comprises a target-specific sequence capable of capturing at least one SNP in the plurality of the SNPs identified across the one or more target chromosome arms. In one example, each primer of the plurality of forward and reverse primer pairs disclosed in step (b)(I) comprises a target-specific sequence capable of capturing at least one SNP, or at least two SNPs, or at least three SNPs, or at least four SNPs, or at least five SNPs, or at least six SNPs, or at least seven SNPs, or at least eight SNPs, or at least nine SNPs, or at least ten SNPs, or at least one hundred SNPs. In one example, each primer of the plurality of forward and reverse primer pairs disclosed in step (b)(II) comprises a target-specific sequence capable of capturing at least one SNP in the plurality of the SNPs identified across one or more target genes. In one example, each primer of the plurality of forward and reverse primer pairs disclosed in step (b)(II) comprises a target-specific sequence capable of capturing at least one SNP, or at least two SNPs, or at least three SNPs, or at least four SNPs, or at least five SNPs, or at least six SNPs, or at least seven SNPs, or at least eight SNPs, or at least nine SNPs, or at least ten SNPs, or at least one hundred SNPs.

In one example, the forward primer and/or reverse primer of the plurality of forward and reverse primer pairs as disclosed herein comprise(s) a “barcode sequence”. As used herein, the term “barcode sequence” refers to an encoded molecule or barcode that includes variable amount of information within the nucleic acid sequence. For example, the barcode sequence is a tag that can be read out using any of a variety of sequence identification techniques, for example, nucleic acid sequencing, probe hybridization-based assay, and the like. The barcode sequence allows the pooled analysis of multiple unique target sequences, where the resulting sequence information from the pool can be later attributed back to each starting target sequence. That is, after the process of amplification, the barcode sequence is used to group amplicons to form a family of amplicons having the same barcode sequence. In some examples, the barcode sequence is an overhang that does not complement any sequence within the target region. As each forward primer carries on its 5′ end a randomly assigned barcode sequence as disclosed herein, the barcode sequence allows individual DNA (such as cfDNA) molecules to be tagged uniquely in the step of sequencing library formation. In one example, the presence of a barcode sequence in each forward primer and each reverse primer of the plurality of forward and reverse primer pairs allows for a more sensitive detection of the nucleic acid sequence.

In one example, each forward primer of the plurality of forward and reverse primer pairs disclosed in step (b)(I) comprises a barcode sequence on the 5′ end (upstream) of the target-specific sequence. In one example, each reverse primer of the plurality of forward and reverse primer pairs disclosed in step (b)(I) comprises a barcode sequence on the 5′ end of the target-specific sequence. In one example, each forward primer or reverse primer of the plurality of forward and reverse primer pairs disclosed in step (b)(I) comprises a barcode sequence on the 5′ end of the target-specific sequence. In one example, each forward primer and reverse primer of the plurality of forward and reverse primer pairs disclosed in step (b)(I) comprise a barcode sequence on the 5′ end of the target-specific sequence. In one example, each forward primer of the plurality of forward and reverse primer pairs disclosed in step (b)(II) comprises a barcode sequence on the 5′ end (upstream) of the target-specific sequence. In one example, each reverse primer of the plurality of forward and reverse primer pairs disclosed in step (b)(II) comprises a barcode sequence on the 5′ end of the target-specific sequence. In one example, each forward primer or reverse primer of the plurality of forward and reverse primer pairs disclosed in step (b)(II) comprises a barcode sequence on the 5′ end of the target-specific sequence. In one example, each forward primer and reverse primer of the plurality of forward and reverse primer pairs disclosed in step (b)(II) comprise a barcode sequence on the 5′ end of the target-specific sequence.

In one example, the barcode sequence is an oligonucleotide comprising 10 to 16 random nucleotides, or 10 to 15 random nucleotides, or 10 to 13 random nucleotides, or 10 random nucleotides, or 11 random nucleotides, or 12 random nucleotides, or 13 random nucleotides, or 14 random nucleotides, or 15 random nucleotides, or 16 random nucleotides. In one example, the barcode sequence is an oligonucleotide comprising 10 to 16 random nucleotides. In one example, the barcode sequence is an oligonucleotide comprising 10 random nucleotides. In one specific example, the barcode sequence is an oligonucleotide comprising 10 random nucleotides which can be represented as NNNNNNNNNN (SEQ ID NO: 1).

In one example, each primer of the plurality of forward and reverse primer pairs disclosed in step (b)(I) comprise an adapter-specific sequence. In one example, each primer of the plurality of forward and reverse primer pairs disclosed in step (b)(II) comprises an adapter-specific sequence. As used herein, the term “adapter-specific sequence” refers to an oligonucleotide sequence bound to the 5′ of the forward primer and/or the 5′ end of the reverse primer. The adapter-specific sequence may be a full adapter-specific sequence or a partial adapter-specific sequence. The adapter-specific sequences are complementary to the plurality of oligonucleotides present on the surface of flow cells of the sequencing tools thereby allowing the nucleic acid fragment (such as DNA fragment or amplicon) to attach to the sequencing tools. The sequencing tools may be any tools, platforms or software known in the art, such as Illumina sequencing. Examples of partial adapter-specific sequences that may be used in Illumina sequencing may include, but are not limited to, 5′-ACACGACGCTCTTCCGATCT-3′ (SEQ ID NO: 2) and 5′-GACGTGTGCTCTTCCGATC-3′ (SEQ ID NO: 3). Examples of full adapter-specific sequences that may be used in Illumina sequencing may include, but are not limited to, 5′-AATGATACGGCGACCACCGAGATCTACACCTAGCGCTACACTCTTTCCCTACACG ACGCTCTTCCGATCT-3′ (SEQ ID NO: 4) and 5′-CAAGCAGAAGACGGCATACGAGATAACCGCGGGTGACTGGAGTTCAGACGTGTG CTCTTCCGATCT-3′ (SEQ ID NO: 5).

The plurality of multiplexed PCR reactions in step (b) generates a plurality of amplicons. In one example, the length of the plurality of amplicons generated in step (b) is 100 to 250 base pairs. In one example, the length of the plurality of amplicons generated in step (b) is less than 100 base pairs. In one example, the length of the plurality of amplicons generated in step (b) is more than 250 base pairs. In one example, the length of the plurality of amplicons generated in step (b) is 110 to 240 base pairs, or 120 to 230 base pairs, or 120 to 220 base pairs, or 130 to 220 base pairs, or 140 to 210 base pairs, or 150 to 200 base pairs, or 160 to 190 base pairs, or 170 to 180 base pairs. In one example, the length of the plurality of amplicons generated in step (b) is 120 to 220 base pairs. The length of the amplicons are optimised to maximise the capture of DNA (such as cfDNA fragments), which range, for example, between 120 to 220 base pairs with a maximum peak at 167 base pairs. In one example, the length of the plurality of amplicons generated in step (b) is about 100 base pairs, or about 110 base pairs, or about 120 base pairs, or about 130 base pairs, or about 140 base pairs, or about 150 base pairs, or about 160 base pairs, or about 170 base pairs, or about 180 base pairs, or about 190 base pairs, or about 200 base pairs, or about 210 base pairs, or about 220 base pairs, or about 230 base pairs, or about 240 base pairs, or about 250 base pairs. In one example, the length of the plurality of amplicons generated in step (b) is about 167 base pairs.

The plurality of amplicons generated in step (b) are then used to generate a plurality of sequencing reads with a next-generation sequencing platform as disclosed in step (c) of the first aspect. The generation of the sequencing reads involves amplification using universal indexed adapter primers (to introduce sample indexes and Illumina sequencing adapters). In one example, the universal indexed adapter primers for use in step (c) of the method of the first aspect comprise:

a forward primer comprising the sequence of
(SEQ ID NO: 6)
AATGATACGGCGACCACCGAGATCTACACCTAGCGCTACACTCTTTCCCT
ACACGACGCTCTTCCGATC*T;
and
a reverse primer comprising the sequence of
(SEQ ID NO: 7)
CAAGCAGAAGACGGCATACGAGATAACCGCGGGTGACTGGAGTTCAGACG
TGTGCTCTTCCGATC*T,,

wherein “*” represents a phosphorothioate bond.

The amplified products are then sequenced on a next-generation sequencing platform to obtain the plurality of sequencing reads. In one example, the plurality of sequencing library is sequenced on NextSeq 550, NextSeq 2000, NovaSeq 6000, BGI MGISEQ-2000, DNBSEQ-G400, or DNBSEQ-T7.

In one example, the plurality of the amplicons generated in step (b) are purified prior to being used to generate a plurality of sequencing reads in step (c). The purification of the amplicons can be performed by using any method or agent known in the art, such as paramagnetic beads selected from a group consisting of AMPure XP beads, SPRI beads, and Dynabeads. In one example, the paramagnetic beads are AMPure XP beads. In one example, the plurality of amplicons generated in step (b) may be treated with enzymes before and/or after the purification of the amplicons to enzymatically digest or remove excess primers. In one example, the enzymes are exonucleases or endonucleases. In one example, the enzymes are exonucleases. In one example, the exonucleases may include, but are not limited to, thermolabile exonuclease I, exonuclease T and exonuclease VII. In one example, the enzymes are endonucleases. In one example, the endonucleases may include, but are not limited to, mung bean nuclease, nuclease P1 and nuclease S1.

The plurality of sequencing reads obtained in step (c) is then used to derive a consensus sequence read of each sequence as disclosed in step (d) of the first aspect. As used herein, the term “consensus sequence read” refers to a nucleotide sequence obtained from consensus calling. In one example, consensus calling is performed by identifying the nucleotide at each position for each sequencing result within the subgroup, comparing the identity for the nucleotide at each position across the plurality of sequencing results, and determining a majority nucleotide at each position. If the majority nucleotide count is above a threshold set for determining majority for specific position, the assignment for said position is the majority nucleotide. If the majority nucleotide count is below this threshold, no assignment is made for said position. The threshold is variable for every position and is a function of the total number of sequencing results corresponding to a specific position.

A sequence alignment is then performed on the consensus reads obtained from step (d) to a reference genome as disclosed in step (e) of the first aspect. As used herein, the term “reference genome” refers to DNA sequences known in the art that may be obtainable from public databases. In one example, the sequence alignment is performed using a sequence alignment tool such as STAR, HISAT2, bwa, CLC, RSEM, kallisto, salmon, etc.

After the sequence alignment in step (e), variant calling is performed in order to calculate variant allele frequency (VAF) as disclosed in step (f) of the first aspect. Variant calling is a process of identifying SNPs or small variants in a single nucleotide within a DNA sequence (such as substitution, insertion, or deletion). The variant calling may be performed using any method known in the art which may include, but is not limited to, a custom variant caller, such as MuTect2, LoFreq and VarScan. As used herein, the term “variant allele frequency (VAF)” is a measurement of genetic variation and may be calculated by dividing the number of variant reads over the number of total reads. VAF is typically reported as a percentage. VAF may be used to provide information on homozygosity and heterozygosity of a locus within the genome. For example, in a normal or a diploid state (i.e., copy number of 2), VAF for a homozygous SNP is about 100% whereas VAF for a heterozygous SNP is about 50%. However, in an abnormal state (such as when LOH is present), the VAF measured may be different from the VAF in a normal or diploid state.

Based on the VAF obtained in step (f), a plurality of informative polymorphic sites is determined and enumerated as disclosed in step (g) of the first aspect. As used herein, an “informative polymorphic site” is defined as a site or locus within the target chromosome arm or target gene that comprises between 5% and 95% VAF. In one example, the range of 5% to 95% VAF indicates the presence of a “heterozygous SNP” within the informative polymorphic site. The term “informative polymorphic site” may be used interchangeably with “informative SNP site” or “heterozygous informative SNP site”. In one example, an informative polymorphic site comprises between 5% and 95% VAF, or 10% to 80% VAF, or 20% to 70% VAF, or 30% to 60% VAF, or 40 to 50% VAF, or 45 to 55% VAF. In one example, an informative polymorphic site comprising between 45% to 55% VAF (such as 45.7-54.1% VAF) refers to the range of a heterozygous SNP for which there is no signature of genetic instability observed. In one example, an informative polymorphic site comprising between 45% to 55% VAF (such as 45.7-54.1% VAF) refers to the range of a heterozygous SNP for which there is no LOH observed. In another example, a VAF falling outside the range of 45% to 55% but is still within the range of 5% to 95% indicates a heterozygous SNP for which one or more signatures of genetic instability is observed. In yet another example, a VAF falling outside the range of 45% to 55% but is still within the range of 5% to 95% indicates a heterozygous SNP for which LOH is observed.

Upon determining and enumerating the plurality of informative polymorphic sites, the allelic ratio (AR) is calculated at each informative polymorphic site as disclosed in step (h) of the first aspect. AR is defined as a ratio of a major allele A to a minor allele B. The AR is then used to classify whether each informative polymorphic site is “genetically unstable” or “genetically stable” (not genetically unstable). In one example, if the AR at each informative polymorphic site is equal to or higher than a pre-determined threshold value, said informative polymorphic site is classified as “genetically unstable”. In another example, if the AR at each informative polymorphic site is lower than a pre-determined threshold value, said informative polymorphic site is classified as “genetically stable” (not genetically unstable)”. In one example, the threshold value, or limit of detection, is determined empirically in a separate manner for each of the signatures of genetic instability, LOH, LST and TAI. A person skilled in the art would be able to determine the threshold value empirically for each of the signatures of genetic instability based on the method as disclosed herein. In one example, the pre-determined AR threshold value for LOH is denoted by the arbitrary variable “χ” for a panel comprising the plurality of forward and reverse primer pairs as disclosed in step (b)(I) and/or step (b)(II) of the first aspect. In one example, the pre-determined AR threshold value for LOH is χ for a panel comprising the plurality of forward and reverse primer pairs as disclosed in step (b)(I) of the first aspect. In one example, the pre-determined AR threshold value for LOH is z for a panel comprising the plurality of forward and reverse primer pairs as disclosed in step (b)(II) of the first aspect. In one aspect, the pre-determined AR threshold value for LOH is χ for a panel comprising the plurality of forward and reverse primer pairs as disclosed in step (b)(I) and step (b)(II) of the first aspect. In one example, the informative polymorphic site is classified as “genetically unstable” for LOH if the AR is equal or greater than χ. In one example, the informative polymorphic site is classified as “genetically stable” (not genetically unstable) for LOH if the AR is less than χ.

The target chromosome arms and/or the target genes are then further determined as to whether they are “positive” for one or more signatures of genetic instability, as disclosed in step (i) of the first aspect. In one example, if the target chromosome arm comprises a minimum pre-determined number of informative polymorphic sites obtained from step (g) and if at least 50% of the informative polymorphic sites are classified as “genetically unstable” in step (h)(I), said target chromosome arm is determined to be “positive” for one or more signatures of genetic instability at chromosome-level. In one example, “at least 50% of the informative polymorphic sites” may include at least 1 out of 2 informative polymorphic sites, or at least 2 out of 3 informative polymorphic sites, or at least 2 out of 4 informative polymorphic sites, or at least 3 out of 4 informative polymorphic sites, or at least 3 out of 5 informative polymorphic sites, or at least 3 out of 6 informative polymorphic sites, or at least 4 out of 5 informative polymorphic sites, or at least 4 out of 6 informative polymorphic sites, or at least 4 out of 7 informative polymorphic sites, or at least 4 out of 8 informative polymorphic sites, etc. In one example, the minimum pre-determined number of informative polymorphic sites for each target chromosome arm to be determined as “positive” is 2, 3, or 4, or 5, or 6, or 7, or 8, or 9, or 10, or 11, or 12, or 13, or 14, or 15. In one example, the minimum pre-determined number of informative polymorphic sites for each target chromosome arm to be determined as “positive” is 4. In one example, if the target gene comprises a minimum pre-determined number of informative polymorphic sites obtained from step (g) and if at least 30% of the informative polymorphic sites are classified as “genetically unstable” in step (h)(I), said target gene is determined to be “positive” for one or more signatures of genetic instability at gene-level. In one example, “at least 30% of the informative polymorphic sites” may include at least 1 out of 2 informative polymorphic sites, or at least 1 out 3 informative polymorphic sites, or at least 2 out of 3 informative polymorphic site, or at least 2 out of 4 informative polymorphic sites, or at least 2 out of 5 informative polymorphic sites, or at least 2 out of 6 informative polymorphic site, or at least 3 out of 4 informative polymorphic sites, or at least 3 out of 5 informative polymorphic sites, or at least 3 out of 6 informative polymorphic sites, or at least 3 out of 7 informative polymorphic sites, or at least 3 out of 8 informative polymorphic sites, or at least 3 out of 9 informative polymorphic sites, etc. In one example, the minimum pre-determined number of informative polymorphic sites for each target gene to be determined as “positive” is 2, 3, or 4, or 5, or 6, or 7, or 8, or 9, or 10, or 11, or 12, or 13, or 14, or 15. In one example, the minimum pre-determined number of informative polymorphic sites for each target gene to be determined as “positive” is 3. In one example, if the one or more target chromosome arms and/or the one or more target genes are determined to be “positive”, then one or more signatures of genomic instability are determined to be present at chromosome-level and/or gene-level within the nucleic acid sample. In one example, if the one or more target chromosome arms are determined to be “positive”, then one or more signatures of genomic instability are determined to be present at chromosome-level within the nucleic acid sample. In one example, if the one or more target genes are determined to be “positive”, then one or more signatures of genomic instability are determined to be present at gene-level within the nucleic acid sample. In one example, if the one or more target chromosome arms and the one or more target genes are determined to be “positive”, then one or more signatures of genomic instability are determined to be present at chromosome-level and gene-level within the nucleic acid sample. In one example, if there is no target chromosome arm and/or target gene that is determined to be “positive”, then one or more signatures of genomic instability are determined to be absent at chromosome-level and/or gene-level within the nucleic acid sample. In one example, if there is no target chromosome arm that is determined to be “positive”, then one or more signatures of genomic instability are determined to be absent at chromosome-level within the nucleic acid sample. In one example, if there is no target gene that is determined to be “positive”, then one or more signatures of genomic instability are determined to be absent at gene-level within the nucleic acid sample. In one example, if there are no target chromosome arm and target gene that is determined to be “positive”, then one or more signatures of genomic instability are determined to be absent at chromosome-level and gene-level within the nucleic acid sample.

In one example, the method of the present disclosure further comprises determining whether the one or more signatures of instability are associated with allelic copy number alteration by:
(j) enumerating the number of allelic copies at the plurality of informative polymorphic sites, wherein if the plurality of informative polymorphic sites are classified as “genetically unstable” in step (h)(I) of the first aspect and
   (I) if there is a decrease (loss) in the number of allelic copies, the one or more signatures of genetic instability are determined to be “copy-number-loss signature”;
   (II) if there is an increase (gain) in the number of allelic copies, the one or more signatures of genetic instability are determined to be “copy-number-gain signature”; and
   (III) if there is no change in the number of allelic copies, the one or more signatures of genetic instability are determined to be “copy-neutral signature”.

In one example, the method of the present disclosure further comprises determining whether the LOH and/or TAI are associated with allelic copy number alteration by:
(j1) enumerating the number of allelic copies at the plurality of informative polymorphic sites, wherein if the plurality of informative polymorphic sites are classified as “genetically unstable” in step (h)(I) of the first aspect and
   (I) if there is a decrease (loss) in the number of allelic copies, the one or more signatures of genetic instability are determined to be “copy-number-loss signature”;
   (II) if there is an increase (gain) in the number of allelic copies, the one or more signatures of genetic instability are determined to be “copy-number-gain signature”; and
   (III) if there is no change in the number of allelic copies, the one or more signatures of genetic instability are determined to be “copy-neutral signature”.

In one example, LOH is associated with one or more types of allelic copy number alterations, wherein the allelic copy number alterations are copy-number-gain, copy-number-loss and/or copy-number-neutral alterations. In one example, the LOH is associated with copy-number-loss alteration. In one example, the LOH is associated with copy-number-neutral alteration. In one example, the LOH is associated with copy-number-loss alteration and copy-number-neutral alteration. In one example, LOH that is associated with a copy-number-gain alteration is referred to as a “copy-number-gain LOH”. In one example, a LOH that is associated with a copy-number-loss alteration is referred to as a “copy-number-loss LOH (CNL-LOH)”. In one example, a LOH that is not associated with a change in the number of allelic copies (i.e, “copy-neutral”) is referred to as a “copy-neutral LOH (cnLOH)”.

In one example, the method of the present disclosure further comprises determining the presence or absence of one or more signatures of genetic instability at global-level within the nucleic acid sample by:
(k) enumerating the number of target chromosome arms and/or target genes determined to be “positive” for one or more signatures of genetic instability at chromosome-level and/or gene-level in step (i) of the first aspect; and
(l) calculating the percentage of the total number of target chromosome arms and/or target genes determined to be “positive” for one or more signatures of genetic instability obtained from step (k) divided by the total number of target chromosome arms and/or target genes in step (a) of the first aspect.

In one example, the presence or absence of one or more signatures of genetic instability at global level is determined by:
(k1) enumerating the number of target chromosome arms determined to be “positive” for one or more signatures of genetic instability at chromosome-level in step (i)(I) of the first aspect; and
(l1) calculating the percentage of the total number of target chromosome arms determined to be “positive” for one or more signatures of genetic instability obtained from step (k1) divided by the total number of target chromosome arms in step (a) of the first aspect.

In one example, the presence or absence of one or more signatures of genetic instability at global level is determined by:
(k2) enumerating the number of target genes determined to be “positive” for one or more signatures of genetic instability at gene-level in step (i)(II) of the first aspect; and
(l2) calculating the percentage of the total number of target genes determined to be “positive” for one or more signatures of genetic instability obtained from step (k2) divided by the total number of target genes in step (a) of the first aspect.

In one example, the presence or absence of one or more signatures of genetic instability at global level is determined by:
(k3) enumerating the number of target chromosome arms and target genes determined to be “positive” for one or more signatures of genetic instability at chromosome-level and gene-level in step (i) of the first aspect; and
(l3) calculating the percentage of the total number of target chromosome arms and target genes determined to be “positive” for one or more signatures of genetic instability obtained from step (k3) divided by the total number of target chromosome arms and target genes in step (a) of the first aspect.

The minimum number of target chromosome arms required to establish global-level genetic instability is variable and depends on, for example, the number of chromosome arms exhibiting the full signature of genetic instability (such as LOH), the number of non-informative chromosome arms, and the cancer type. In one example, the number of target chromosome arms required to establish global-level genetic instability is at least ten. The minimum number of genes required to establish global-level genetic instability may be dependent on the stage of the disease. In one example, the number of target gene required to establish global-level genetic instability is at least one. In one example, the number of target chromosome arms required to establish chromosome-level LOH is at least one. In one example, the number of target gene required to establish chromosome-level genetic instability is at least one. In one example, the number of target chromosome arms required to establish gene-level LOH is at least one. In one example, the number of target gene required to establish gene-level genetic instability is at least one.

The method of the present disclosure may be used with different types of nucleic acid samples. In one example, the nucleic acid sample is selected from a DNA sample or an RNA sample. In one example, the DNA sample may include, but is not limited to, cell-free DNA (cfDNA) or DNA encapsulated within tissues and/or cells. In one example, the DNA sample is a cfDNA sample. In one example, tumour-derived cfDNA (ctDNA) may be found within the cfDNA sample. In one example, the RNA sample is selected from the group consisting of messenger RNA, circular RNA and non-coding RNA, or RNA encapsulated within tissues and/or cells. In one example, the RNA is converted into DNA prior to step (a) of the method of the first aspect. In one example, the DNA or RNA encapsulated within tissues and/or cells may be extracted first using any method known in the art. In one example, the DNA or RNA is extracted from the tissues and/or cells prior to step (a) of the method of the first aspect. In one example, the tissue may be any type of tissue in the human body. In another example, the cell may be any type of cell in the human body. In one example, the DNA or RNA may be extracted from the tissues and/or cells using any kit known in the art, such as AllPrep DNA/RNA Mini (QIAGEN), QIAamp ccfDNA/RNA Kit (Qiagen), Isopure Plasma cfDNA/RNA Isolation Kit (Aline Biosciences), MagMAX™ Cell-Free Total Nucleic Acid Isolation Kit (Applied Biosystems), QIAamp Circulating Nucleic Acid kit (Qiagen), Zymo Quick-cfRNA Serum & Plasma Kit (Zymo Research), and NextPrep™ Magnazol™ cfRNA Isolation Kit (PerkinElmer), etc.

The method of the present disclosure may be performed using a liquid sample, a tissue sample or a cell sample. In one example, the nucleic acid sample is a liquid sample, a tissue sample, or a cell sample. In one example, the nucleic acid sample is a liquid sample such as a bodily fluid. In one example, the bodily fluid may include, but is not limited to, blood, bone marrow, cerebral spinal fluid, peritoneal fluid, pleural fluid, lymph fluid, ascites, serous fluid, sputum, lacrimal fluid, stool, urine, saliva, ovarian fluid, oviductal fluid, prostatic fluid, ductal fluid from breast, gastric juice and pancreatic juice. In one example, the bodily fluid is blood. In one example, the blood is plasma. The tissue sample may include, but is not limited to, a frozen tissue sample or a fixed tissue sample. In one example, the fixed tissue sample is a Formalin-Fixed Paraffin-Embedded (FFPE) tissue sample. The cell sample may be from any type of cell in the body. In one example, the cell is from bone, epithelial, cartilage, adipose tissue, nerves, muscle, connective tissue, esophagus, stomach, liver, gallbladder, pancreas, adrenal glands, bladder, gallbladder, large intestine, small intestine, kidneys, liver, pancreas, colon, stomach, thymus, spleen, brain, spinal cord, heart, lungs, eyes, corneal, skin, or islet tissue or organs. In one example, the cell may be a cancer cell, a stem cell, an endothelial cell, or a fat cell. In one example, the cell is a blood cell. The blood cell may be a white blood cell, or a platelet. In one example, the cell is selected from a cancer cell. In one example, the cancer cell is associated with a DNA repair deficiency disorder, such as HRD.

In one example, the nucleic acid sample is obtained from a subject having and/or suspected of having a disorder associated with one or more signatures of genetic instability. The disorder associated with one or more signatures of genetic instability may include, but is not limited to, a DNA repair deficiency disorder such as Homologous Recombination Deficiency (HRD), Non-Homologous End-Joining (NHEJ) Deficiency, DNA mismatch repair (MMR) deficiency, nucleotide excision repair (NER) deficiency, and base excision repair (BER) deficiency. In one example, the DNA repair deficiency disorder is HRD.

In one example, the subject has or is suspected of having a DNA repair deficiency disorder if one or more signatures of genetic instability are present at gene-level, chromosome-level and/or global-level within the nucleic acid sample of the subject. In one example, the subject has or is suspected of having a DNA repair deficiency disorder if one or more signatures of genetic instability are present at gene-level within the nucleic acid sample of the subject. In one example, the subject has or is suspected of having a DNA repair deficiency disorder if one or more signatures of genetic instability are present at chromosome-level within the nucleic acid sample of the subject. In one example, the subject has or is suspected of having a DNA repair deficiency disorder if one or more signatures of genetic instability are present at global-level within the nucleic acid sample of the subject. In one example, the subject has or is suspected of having a DNA repair deficiency disorder if one or more signatures of genetic instability are present at gene-level and chromosome-level within the nucleic acid sample of the subject. In one example, the subject has or is suspected of having a DNA repair deficiency disorder if one or more signatures of genetic instability are present at gene-level and global-level within the nucleic acid sample of the subject. In one example, the subject has or is suspected of having a DNA repair deficiency disorder if one or more signatures of genetic instability are present at chromosome-level and global-level within the nucleic acid sample of the subject. In one example, the subject has or is suspected of having a DNA repair deficiency disorder if one or more signatures of genetic instability are present at gene-level, chromosome-level and global-level within the nucleic acid sample of the subject.

In one example, the DNA repair deficiency disorder is associated with a cancer. The cancer may be selected from, but is not limited to, ovarian cancer, prostate cancer, breast cancer, leukaemia, lung cancer, colorectal cancer, pancreatic cancer, nasopharyngeal cancer, liver cancer, cholangiocarcinoma, oesophageal cancer, urothelial cancer, and gastrointestinal cancer, endometrial cancer, peritoneal cancer, cervical cancer, thyroid cancer, kidney cancer, and brain cancer. In one example, the cancer is ovarian cancer. In one example, the cancer is prostate cancer. In one example, the cancer is breast cancer.

In one example, the method of the present disclosure comprises detecting the presence or absence of one or more signatures of genetic instability at chromosome-level and/or gene-level within a cfDNA sample, wherein the method further comprises using the AR ratio obtained from step (h) to determine the fraction of tumour-derived circulating DNA (ctDNA) that may be present within the cfDNA sample. ctDNA is a subset of cfDNA of tumour origin. The determination of the fraction of ctDNA within the cfDNA sample provides information on the presence, progression and/or stages of the cancer as well as tumour burden. In one example, an increase in the fraction of ctDNA within the cfDNA sample indicates the worsening of the cancer. In another example, the higher the fraction of ctDNA within the cfDNA sample, the higher the tumour burden. The information obtained may in turn be used to determine the appropriate anticancer treatment.

In a second aspect, the present disclosure refers to a kit for detecting the presence or absence of one or more signatures of genetic instability at chromosome-level and/or gene-level within a nucleic acid sample according to the method as disclosed herein, wherein the kit comprises a plurality of forward and reverse primer pairs that are capable of capturing a plurality of SNPs identified across a one or more target chromosome arms as defined in step (b)(I) of the method of the first aspect and/or a plurality of forward and reverse primer pairs that are capable of capturing a plurality of SNPs identified across one or more target genes as defined in step (b)(II) of the method of the first aspect. In one example, the kit further comprises instructions for use in the method as disclosed herein. In another example, the kit further comprises: a buffer for performing a plurality of multiplexed PCR reactions, universal indexed adapter primers, a DNA polymerase and a plurality of deoxynucleoside triphosphates (dNTPs). In one example, the kit further comprises an exonuclease. In some examples, the reagents provided in the kit as described herein may be provided in separate containers comprising the components independently distributed in one or more containers. As the method as described herein relates to sequencing (such as high-throughput sequencing), further components required in sequencing process could be easily determined by the person skilled in the art.

The method of the present disclosure may also be used to predict and/or monitor the response of a subject having a disorder associated with one or more signatures of genetic instability towards one or more therapeutic agents, such as poly (ADP-ribose) polymerase inhibitors and platinum-based chemotherapy drugs. In one example, the therapeutic agent is a poly (ADP-ribose) polymerase inhibitor.

In a third aspect, the present disclosure refers to a method of predicting and/or monitoring the response of a subject having a disorder associated with one or more signatures of genetic instability towards treatment with one or more poly (ADP-ribose) polymerase inhibitors, comprising detecting the presence or absence of one or more signatures of genetic instability at chromosome-level and/or gene level according to the method of the first aspect. In one example, if one or more signatures of genetic instability are present in the subject, the subject is predicted to be responsive or more responsive towards the treatment with one or more poly (ADP-ribose) polymerase inhibitors compared to another subject without the one or more signatures of genetic instability. In one example, if one or more signatures of genetic instability are not present in the subject, the subject is predicted to be unresponsive (not responsive) or less responsive towards the treatment with one or more poly (ADP-ribose) polymerase inhibitors compared to another subject with the one or more signatures of genetic instability. In one example, if one or more signatures of genetic instability are still present in the subject after treatment with one or more poly (ADP-ribose) polymerase inhibitors, the subject is not responsive or has not responded to said treatment. In one example, if one or more signatures of genetic instability are absent from the subject after treatment with one or more poly (ADP-ribose) polymerase inhibitors, the subject is responsive or has responded to said treatment. In one example, the response of the subject towards treatment with one or more poly (ADP-ribose) polymerase inhibitors is determined by detecting the presence or absence of one or more signatures of genetic instability at chromosome-level and/or gene level according to the method of the first aspect after 1 month, or after 2 months, or after 3 months, or after 4 months, or after 5 months, or after 6 months, or after 7 months, or after 8 months, or after 9 months, or after 10 months, or after 11 months, or after 12 months, or after 18 months, or after 24 months, or after 30 months, or after 36 months, or after 48 months, or after 60 months, or after 72 months, or after 84 months, or after 96 months of the treatment. In one example, the response of the subject towards treatment with one or more poly (ADP-ribose) polymerase inhibitors is monitored every week, or every 2 weeks, or every 4 weeks, or every 6 weeks, or every 8 weeks, or every 3 months, or every 6 months, or every year, or every 2 years, or every 3 years. In one example, the subject has a DNA repair deficiency disorder such as Homologous Recombination Deficiency (HRD), Non-Homologous End-Joining (NHEJ) Deficiency, DNA mismatch repair (MMR) deficiency, nucleotide excision repair (NER) deficiency, and base excision repair (BER) deficiency. In one example, the subject has HRD. In one example, the poly (ADP-ribose) polymerase inhibitor may include, but is not limited to, rucaparib, olaparib, niraparib, talazoparib, and veliparib.

As used in this application, the singular form “a,” “an,” and “the” include plural references unless the context clearly dictates otherwise. For example, the term “a primer” includes a plurality of primers, including mixtures and combinations thereof.

As used herein, the term “presence” (or grammatical variants thereof) in the context of a feature, trait, characteristic or substance refers to the state of the feature, trait, characteristic or substance being detected, present, or in existence. For example, the “presence” of a signature of genetic instability within a nucleic acid sample indicates that the signature is detected or exists or is present within the nucleic acid sample. As used herein, the term “absence” (or grammatical variants thereof) in the context of a feature, trait, characteristic or substance refers to the state of the feature, trait, characteristic or substance being not detected, not present (absent) or in non-existence. For example, the “absence” of a signature of genetic instability within a nucleic acid sample indicates that the signature is not detected, not present or does not exist within the nucleic acid sample. As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the inventions embodied therein herein disclosed may be resorted to by those skilled in the art, and that such modifications and variations are considered to be within the scope of this present disclosure.

The disclosure has been described broadly and generically herein. Each of the narrower species and subgeneric groupings falling within the generic disclosure also form part of the present disclosure. This includes the generic description of the present disclosure with a proviso or negative limitation removing any subject matter from the genus, regardless of whether or not the excised material is specifically recited herein.

Other embodiments are within the following claims and non-limiting examples.

### Examples

### Materials

Exemplary Molecular Tag Complex or Primers when Target is a SNP in BRCA1 Exon 10

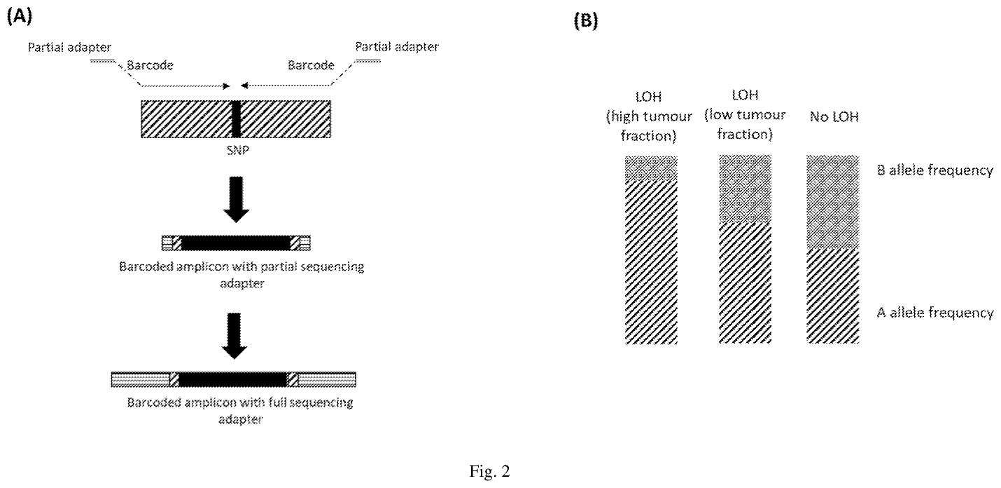

An example of a “primer” when the target sequence is BRCA1_SNP37 (an example of forward target capture primer, illustrated in FIG. 2A is as follows:

(SEQ ID NO: 8)
ACACGACGCTCTTCCGATCTNNNNNNNNNNTCTTCTGAGGACTCTAATTT
CTTGG,

wherein the bases in italic and underline are an example of adapter sequence, the bases in bold represent the barcode sequence and the bases in underline is an example of target specific sequence.

An example of subsequent primers for the “completion of amplicon” (an example of a reverse target capture primer, illustrated also in FIG. 2A) is as follows:

(SEQ ID NO: 9)
GACGTGTGCTCTTCCGATCTNNNNNNNNNNGATACTAGTTTTGCTGAAAA
TGACA,

wherein the bases in italic and underline are an example of adapter sequence, the bases in bold represent the barcode sequence and the bases in underline is an example of target specific sequence.

Expected amplicon (only target-specific region) >chr17:41243907+41244064 158bp

(SEQ ID NO: 10)
TCTTCTGAGGACTCTAATTTCTTGGcccctcttcggtaaccctgagccaa
atgtgtatgggtgaaagggctaggactcctgctaagctctcctttctgga
cgcttttgctaaaaacagcagaactttccttaaTGTCATTTTCAGCAAAA
CTAGTATC

Product after amplicon completion, illustrated also in FIG. 2A (in two steps) (Only one strand of the double stranded product is shown.):

(SEQ ID NO: 11)
ACACGACGCTCTTCCGATCTNNNNNNNNNNTCTTCTGAGGACTCTAATTT
CTTGGcccctcttcggtaaccctgagccaaatgtgtatgggtgaaagggc
taggactcctgctaagctctcctttctggacgcttttgctaaaaacagca
gaactttccttaaTGTCATTTTCAGCAAAACTAGTATCNNNNNNNNNNAG
ATCGGAAGAGCACACGTC,

where the bases in underline is target nucleic acid.

Final product, illustrated also in FIG. 2A (suitable for sequencing on Illumina)

(SEQ ID NO: 12)
AATGATACGGCGACCACCGAGATCTACACCTAGCGCTACACTCTTTCCCT
ACACGACGCTCTTCCGATCTNNNNNNNNNNTCTTCTGAGGACTCTAATTT
CTTGGcccctcttcggtaaccctgagccaaatgtgtatgggtgaaagggc
taggactcctgctaagctctcctttctggacgcttttgctaaaaacagca
gaactttccttaaTGTCATTTTCAGCAAAACTAGTATCNNNNNNNNNNAG
ATCGGAAGAGCACACGTCTGAACTCCAGTCACCCGCGGTTATCTCGTATG
CCGTCTTCTGCTTG,

where the bases in underline is target nucleic acid.

### Methods

### Sample Collection and Processing

Blood collected in Cell-free DNA BCT (Streck) was shipped at ambient temperature before plasma separation. Plasma was prepared using a 2-step centrifugation process: first centrifugation was done at 1600×g for 10 min at 4° C. to separate plasma. The plasma layer was transferred to a separate tube and centrifuged at 16,000×g for 10 min at 4° C. to further remove cellular contaminants, and immediately processed for nucleic acid extraction or stored at −80° C. until used for extraction. If frozen, the plasma was fully thawed at room temperature before extraction.

Cell-free total nucleic acids were extracted from 3-5 mL of plasma using the QIAamp Circulating Nucleic Acid kit (Qiagen). Cell-free DNA (cfDNA) was quantified using the Qubit 1× dsDNA High Sensitivity kit (Thermo Fisher Scientific).

### Design of Primers for Detection of Global and Gene-Specific Loss of Heterozygosity (LOH)

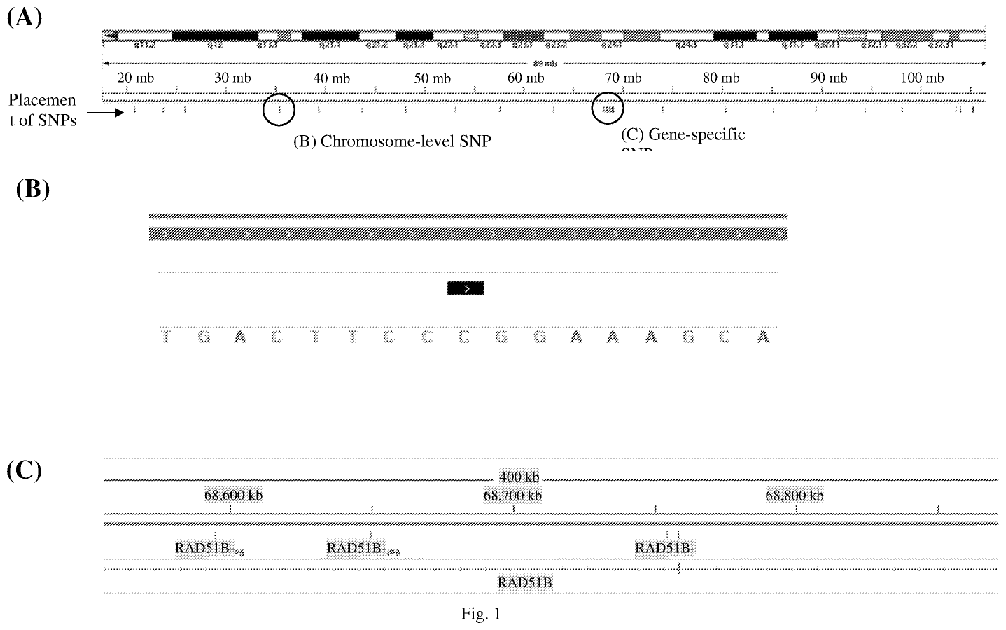

To detect LOH, a highly multiplex amplicon-based NGS assay was designed to capture single nucleotide polymorphisms (SNPs) across the genome (FIG. 1A). Each target capture primer is composed of three parts—the target-specific sequence, a 10-bp random nucleotide sequence (NNNNNNNNNN) upstream of the target-specific sequence, and an adapter-specific sequence. The target-specific sequence achieves target capture, the 10-bp random nucleotide constitutes the “unique molecular barcode”, and the adapter-specific sequence serves as the primer landing site for the final library amplification primers. As explained in the data analysis section below, the combination of the target-specific sequence and the 10-bp unique molecular barcode for both forward and reverse primers is used to trace and define a unique original parental DNA molecule. For detecting global LOH, SNPs were sparsely distributed across each chromosome arm at uniform intervals (FIG. 1B), ranging from 2 to 10 Mb depending on the length of the chromosome arm. For detecting gene-specific LOH, SNPs were densely distributed across key HRR genes to capture gene-specific LOH (FIG. 1C). SNP inclusion was guided by two additional criteria. First, SNPs with low population frequencies (<40% for chromosome-level SNPs and <10% for gene-level SNPs) were excluded. Second, insertion-deletion mutations were excluded, along with single-nucleotide variants found within tandem repeats.

### Preparation of Sequencing Library

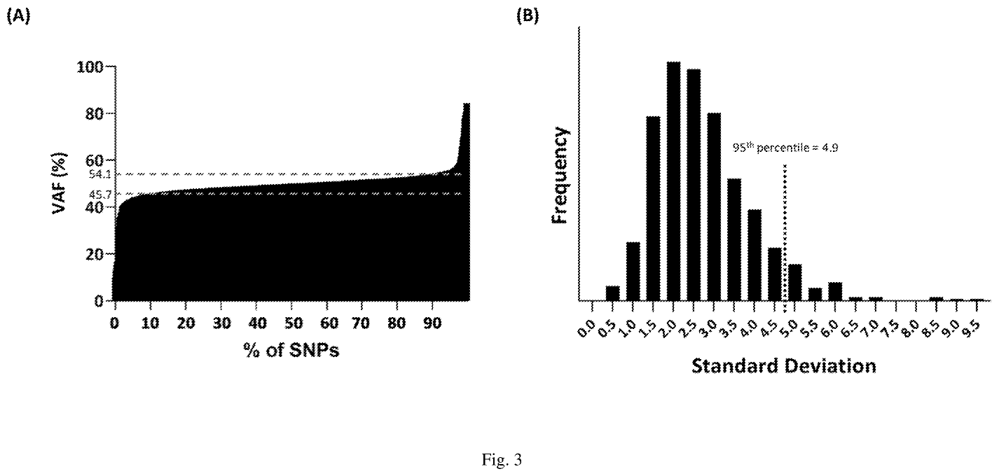

The generation of a sequencing library is achieved in three steps (FIG. 3A): (1) molecular barcode assignment and amplicon generation (multiplex target capture PCR), (2) removal of excess target capture primers (exonuclease treatment), and (3) final library amplification (indexing PCR).

### Molecular Barcode Assignment and Amplicon Generation

In this first step, target DNA molecules are captured with a pair of primers per target. Cell-free DNA was used as a template in a highly multiplexed PCR reaction for target capture using the Platinum™ SuperFi II DNA Polymerase (Thermo Fisher Scientific). Briefly, in a 50 μL PCR reaction, cfDNA was mixed with target capture primers at a final concentration of 10-100 nM (each primer), 10 μL of 5× SuperFi II Buffer, 10 nM dNTPs, and 2 μL Platinum SuperFi II DNA Polymerase, and subjected to the following thermocycling conditions: initial denaturation at 98° C. for 30s; followed by 3-5 cycles of denaturation at 98° C. for 10 s, annealing at 58° C. for 6 mins, extension at 72° C. for 1 min; and lastly a final extension at 72° C. for 5 min.

### Removal of Excess Target Capture Primers

The PCR product underwent exonuclease treatment by adding 6.1 μL 10× NEBuffer r3.1 (NEB), 2.5 μl thermolabile exonuclease I (NEB) and 2.5 μL exonuclease T (NEB), followed by an incubation at 37° C. for 10 min. The exonuclease-treated product was then subjected to clean-up using 1.5× volume of AMPure XP beads (Beckman Coulter), and eluted in 23 μL of Buffer EB (Qiagen).

### Final Library Amplification

Purified products were then amplified with universal indexed adapter primers (to introduce sample indexes and Illumina sequencing adapters) in a 50 μL reaction with 2 μM (final concentration) primers using KAPA HiFi HotStart ReadyMix (Roche). The PCR was carried out with the following thermocycling profile: initial denaturation at 98° C. for 45 s; followed by 14-16 cycles of denaturation at 98° C. for 15 s, annealing at 60° C. for 30 s, extension at 72° C. for 30 s; and lastly a final extension at 72° C. for 1 min. The amplified library was purified with two rounds of 0.8× volume AMPure XP beads to remove excess adapters and size-select the final sequencing library. Each final purified library (FIG. 2B) was qualified using the High Sensitivity DNA Screentape (Agilent) and quantified using KAPA Library Quantification Kit (Roche) before being sequenced on a NextSeq 550 system (Illumina).

### Data Analysis

Binary base call sequencing files were first demultiplexed and converted to FASTQ files, which were processed using a custom pipeline. First, bases with poor quality scores were filtered. Next, read 1 and corresponding read 2 FASTQ files were searched for expected forward and reverse primer sequences respectively, based on an input file containing named primer sequences of all amplicons within the panel. Primer sequences and upstream molecular barcode sequences were trimmed using cutadapt and the trimmed sequences were mapped to the reference genome using bwa-mem. Reads were annotated with their corresponding primer names. The primer name assigned to read 1 may not always match that of read 2 due to overlapping amplicons or non-specific binding. An “amplicon_name” was assigned to each read pair by concatenating the matching primer name of reads 1 and 2 (F_name;R_name). Molecular barcode sequences from both reads 1 and 2 were also concatenated and assigned separately to each paired read (F_barcode;R_barcode).

Subgraph consensus clustering of molecular barcodes was performed by considering each amplicon_name as a network. Each read assigned the same amplicon_name was represented within the amplicon_name network as a subgraph of 2 connected nodes of identity F_barcode and R_barcode. Every subsequent read was added to the network either as a disconnected subgraph or joined to an existing subgraph via a common barcode (either F_barcode or R_barcode), until no more reads are left. Each consensus cluster was a disconnected subgraph within the network and is represented by the amplicon_name appended with a number (amplicon_name_n). Consensus clusters with fewer than 1-5 members were considered unreliable and removed prior to downstream analyses.

Consensus calling was done for each consensus cluster, first via global alignment of all consensus family members using MAFFT. The consensus base in each aligned position was called by determining the majority representative base, the percentage of which is no less than an automatically determined threshold, which is a function of the total number of reads within the consensus cluster. If no representative base could be called, the position was assigned N, as opposed to one of A, C, T, G. A new quality score was assigned to each position, which is either 90th percentile of all the quality values from the representative base type in that position if a consensus base is found, or 10th percentile of all quality values in that position if no consensus base is found. The consensus reads were written to new consensus FASTQ files, which were then mapped to the reference genome with local realignment to improve mapping. Consensus read depth was calculated from the mapped BAM file as the unique number of consensus clusters mapped to each target region specified in the panel. Variant calling was performed on consensus BAM files using a custom variant caller.

All single nucleotide variants between 5 and 95% variant allele frequency (VAF) and possessing a dbSNP and gnomAD entry were considered as informative polymorphic sites for LOH determination. Allelic ratio (AR) at each informative polymorphic site was calculated as the ratio of major (A) to minor (B) allele. Each informative polymorphic site was classified as ‘LOH’ if the AR was ≥χ, and ‘no LOH’ if AR<χ. Gene-specific LOH was established when a minimum of 3 informative gene-specific SNPs was available, of which at least 30% of informative polymorphic sites were scored as ‘LOH’. The global LOH signature was evaluated on the chromosome arm level. Chromosome arms with a minimum of 4 informative polymorphic sites and at least 50% of informative polymorphic sites presenting with LOH were considered as ‘LOH positive’. Because gene-specific LOH amplicons were densely packed and provide only localised information, these informative polymorphic sites were aggregated as a single AR at the gene level in the determination of global LOH. Global LOH was scored as a percentage of the number of ‘LOH positive’ chromosome arms/total number of chromosome arms for consideration, where total number of arms for consideration can be maximum of 39 (22*2 autosomal chromosomes, excluding the p arms from 5 acrocentric chromosomes 13, 14, 15, 21, 22 each), and excludes chromosome arms with insufficient informative polymorphic sites (cannot be confirmed to be LOH-negative) or where the entire arm length exhibits LOH.

### Results

To detect LOH, a targeted multiplex amplicon-based NGS panel for the detection of single nucleotide polymorphisms (SNPs) across the genome was designed (FIG. 1A). Amplicon lengths were optimised to maximise capture of cfDNA fragments, which typically range between 120-220 bp with a maximum peak at 167 bp. Separate approaches were used for SNP placement to capture 2 types of information. First, SNPs were sparsely distributed across each chromosome arm at uniform intervals (FIG. 1B), ranging from 2-10 Mb depending on the length of the chromosome arm, to capture chromosome-level LOH. Second, SNPs were densely distributed across key HRR genes to capture gene-specific LOH (FIG. 1C). Examples of targeted HRR genes include those listed in Table 1. SNP recruitment was guided by two additional criteria. First, SNPs with low population frequencies (<40% for chromosome-level SNPs and <10% for gene-level SNPs) were excluded. Second, insertion-deletion mutations were excluded, along with single-nucleotide variants found within tandem repeats. This approach maximises both the number of informative polymorphic sites as well as enables higher accuracy during the enumeration of unique DNA copies.

TABLE 1
Selected homologous recombination repair (HRR) pathway genes:

| Gene | Full name of gene |
|---|---|
| ARID1A | AT-rich interaction domain 1A |
| ATM | ATM serine/threonine kinase |
| ATR | ATR serine/threonine kinase |
| ATRX | ATRX chromatin remodeler |
| BAP1 | BRCA1 associated protein 1 |
| BARD1 | BRCA1 associated RING domain 1 |
| BLM | BLM RecQ like helicase |
| BRCA1 | BRCA1 DNA repair associated |
| BRCA2 | BRCA2 DNA repair associated |
| BRIP1 | BRCA1 interacting helicase 1 |
| CDK12 | cyclin dependent kinase 12 |
| CHEK1 | Checkpoint kinase 1 |
| CHEK2 | Checkpoint kinase 2 |
| EMSY | EMSY transcriptional repressor, BRCA2 interacting |
| FANCA | FA complementation group A |
| FANCC | FA complementation group C |
| FANCD2 | FA complementation group D2 |
| FANCE | FA complementation group E |
| FANCF | FA complementation group F |
| FANCG | FA complementation group G |
| FANCI | FA complementation group I |
| FANCL | FA complementation group L |
| FANCM | FA complementation group M |
| MRE11 | MRE11 homolog, double strand break repair nuclease |
| NBN | nibrin |
| PALB2 | Partner and localizer of BRCA2 |
| PTEN | Phosphatase and tensin homolog |
| RAD50 | RAD50 double strand break repair protein |
| RAD51 | RAD51 recombinase |
| RAD51B | RAD51 paralog B |
| RAD51C | RAD51 paralog C |
| RAD51D | RAD51 paralog D |
| RAD52 | RAD52 homolog, DNA repair protein |
| RAD54L | RAD54 like |
| RPA1 | Replication protein A1 |
| XRCC2 | X-ray repair cross complementing 2 |

Each forward and reverse primer in the multiplex panel contains molecular barcodes (FIG. 2A), which enable accurate and reproducible enumeration of unique DNA copies. The utility of this molecular barcoding approach is two-fold. First, it enables accurate enumeration of unique DNA molecules, which is required both for the determination of variant allele frequencies (VAFs) as well as DNA copy number changes. Second, it enables highly efficient recovery of template DNA molecules, circumventing the issues presented with cfDNA regarding low ctDNA content and low cfDNA amounts in plasma.

LOH is assessed at each heterozygous SNP position by comparing the allelic ratio (AR) of the major (A allele) and minor (B allele) alleles. At normal heterozygous genomic loci with no LOH, the ratio of the major to minor allele is 1. Deviation of this ratio from 1 indicates loss of heterozygosity; in a DNA sample with 100% tumour purity, only one allele is present (AR=100/0). As cfDNA is a mixture of DNA of tumour (ctDNA) and normal (gDNA) origin, the AR is directly dependent on the fraction of ctDNA in cfDNA, referred to as the tumour fraction (TF), and can take any value≥1. Thus, the magnitude of AR can be used to evaluate both the presence of LOH as well as the tumour fraction of a cfDNA sample (FIG. 2B).

Accuracy of measurement by the panel was established via sequencing of 2.5 ng of 8 genomic DNA (gDNA) samples (normal DNA with no known LOH). Analysis of all SNPs across 10-90% variant allele frequency (VAF) (excluding low-level noise from sequencing and homozygous SNPs with ˜100% VAF) gave a median VAF of 50.0%, with 90% of SNPs falling between 45.7%-54.1% VAF (FIG. 3A). Initial assessment of the limit of detection for LOH, based on the precision of the panel, was made by sequencing 5-10 NGS library replicates of 5 cfDNA samples. Analysis of 693 heterozygous SNPs indicated that 95% of SNP replicates deviated no more than 4.9% from mean VAFs (FIG. 3B). Given this, the limit of detection of LOH based on AR was preliminarily established, accounting for the methodological limits in the measurement precision of VAFs by this NGS panel.

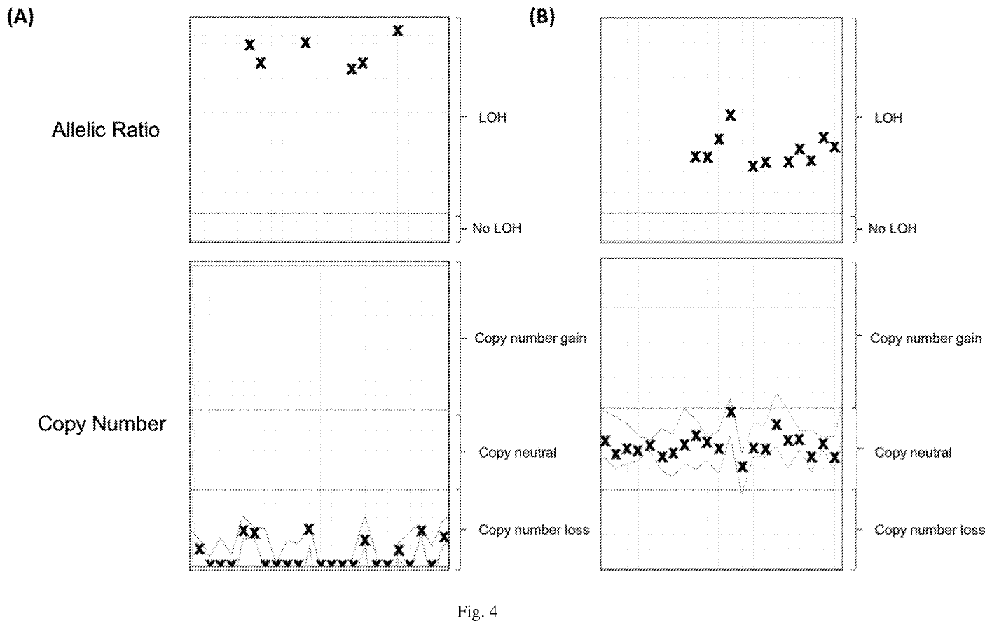

Two main types of LOH exist, LOH with copy number loss (CNL-LOH), and copy-neutral LOH (cnLOH). To differentiate between the two, AR calculation is combined with total copy number enumeration. In CNL-LOH, deviated AR is coupled with a loss in copy number (FIG. 4A), while in cnLOH, only a deviation of AR is observed (FIG. 4B). Although the AR threshold does not change for the two types of LOH, the presence of accompanying copy number loss in CNL-LOH means that the limit of detection expressed in tumour fractions is different for CNL-LOH and cnLOH.

Assuming a normal diploid copy number, copy number=2 in normal DNA and copy number=1 in tumour DNA for CNL-LOH. Hence,

$$A R = \frac{A allele ( tumour DNA + n ormal DNA )}{B allele ( normal DNA )} = \frac{T F + ( 1 - T F )}{1 - T F} = \frac{1}{1 - T F}$$

$$and$$

$$TF = 1 - \frac{1}{A R} .$$

For cnLOH, copy number=2 in both normal and tumour DNA. Hence,

$$AR = \frac{A allele ( tumour DNA + n ormal DNA )}{B allele ( normal DNA )} = \frac{2 * T F + ( 1 - T F )}{1 - T F} = \frac{1 + T F}{1 - T F}$$

$$and$$

$$TF = \frac{AR - 1}{AR + 1} .$$

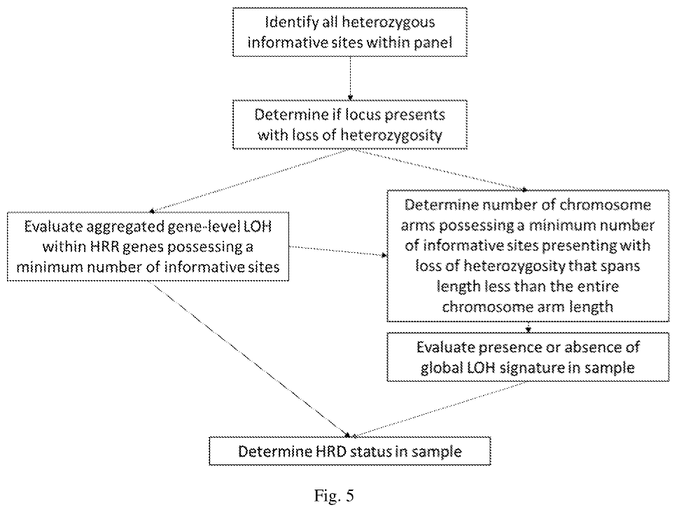

To generate high confidence LOH calls, multiple concordant calls from distinct amplicons within the same target are required. The ability to call LOH relies on the presence of informative SNPs in a particular sample in the genomic/gene regions being interrogated. An informative SNP is defined as one which has both A and B alleles represented, i.e. it is heterozygous under conditions of no LOH. In this method, gene-specific LOH is established when a minimum of 3 informative gene-specific SNPs is available, of which at least 30% of informative polymorphic sites are scored as LOH positive, based on the allelic ratio at specific SNPs. Separately, the global LOH signature is evaluated on the chromosome arm level. Chromosome arms with a minimum of 4 informative polymorphic sites and at least 50% of informative polymorphic sites presenting with LOH are considered as LOH positive. Because gene-specific LOH amplicons are densely packed and provide only localised information, these informative polymorphic sites are aggregated as a single AR at the gene level in the determination of global LOH. Chromosome arms where the entire arm length exhibits LOH are excluded from consideration as these are likely to originate from alternative mechanisms not involving homologous recombination repair. Together, gene-specific LOH and global LOH calls are used to evaluate the HRD status in a given sample (FIG. 5).

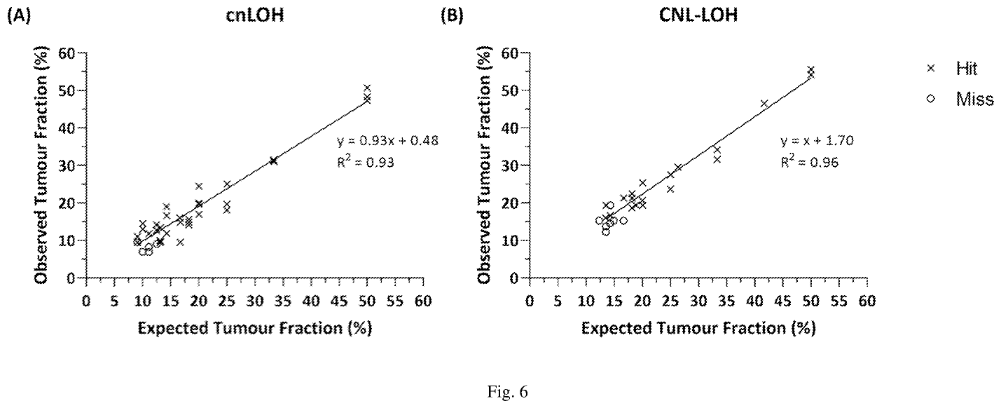

The ability of the panel to detect gene-level CNL-LOH and cnLOH was confirmed by sequencing DNA from cell-lines known to harbour LOH (HCC1395 and HCC1937, representing tumour DNA) admixed with their respective EBV-immortalised peripheral blood lymphocyte cell-line DNA (HCC1395BL and HCC1937BL, representing normal DNA without LOH). Admixtures were generated to produce tumour fractions ranging from 9% to 50%. Copy-neutral LOH as low as 10% TF (FIG. 6A) and CNL-LOH as low as 18% (FIG. 6B) could be detected using 2.5 ng of admixed DNA. In addition, the accurate estimation of TF at each tested TF is demonstrated (R² of observed against expected TF≥0.93).

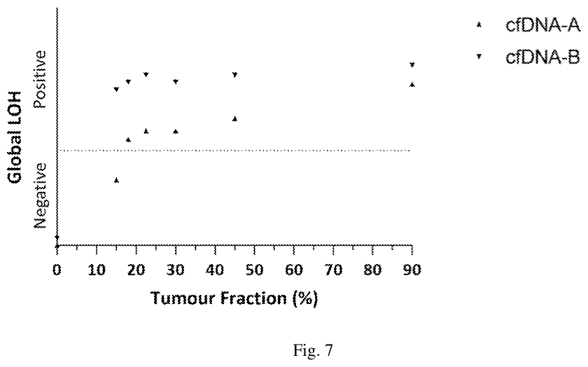

The ability of the panel to detect the presence of global LOH signature in cfDNA was confirmed by sequencing two cfDNA samples with known HRD positivity (based on mutations in HRR genes and tissue-matched HRD score) and tumour fractions of ˜90%. Admixing of cfDNA samples with their corresponding buffy coat gDNA was performed in silico to produce tumour fractions ranging from 15%-45%. In both samples, the global LOH signature could be detected at tumour fractions≥18%. Additionally, the absence of the global LOH signature was confirmed in the respective buffy coat gDNA samples (FIG. 7).

Because fixed tissue DNA does not pose a significantly different challenge from cfDNA for the detection of LOH, it is anticipated that this method will similarly be suitable for the detection of global and gene-specific LOH in tissue DNA. To illustrate this, 2.5 ng of tissue DNA from 46 samples were sequenced and compared against genomic instability calls made using a commercially validated tissue HRD panel which uses 50 ng of tissue DNA input and an NGS panel encompassing>20 000 SNPs (13). An overall concordance of 91.3% (95% CI, 79.7%-96.6%) was established, including 94.4% (95% CI, 81.9%-99.0%) positive percent agreement and 80.0% (95% CI, 49.0%-96.5%) negative percent agreement (Table 2), demonstrating not only broad equivalency in tissue DNA, but suitability with cfDNA where low DNA inputs may be necessary.

TABLE 2
Comparison of genomic instability calls from the method
of the present disclosure against a commercial validated
tissue panel in 50 tissue DNA samples.

| | Commercial method + | Commercial method − |
|---|---|---|
| Current method + | 34 | 2 |
| Current method − | 2 | 8 |

Based on Table 2, the overall percent agreement (OPA) is 91.3% (79.7%-96.6%), positive percent agreement (PPA) is 94.4% (81.9%-99.0%), negative percent agreement (NPA) is 80.0% (49.0%-96.5%).

Hence, in the present disclosure it is shown that genomic instability as evidenced using a global LOH signature as well as gene-specific LOH can be detected using cfDNA from plasma or other biological fluids as an analyte, as well as tissue DNA. The target gene coverage for gene-specific LOH can be expanded in this multiplex NGS via the addition of primers following the same primer design methodology as disclosed herein.

### Discussion

In one example, a method to detect LOH in cfDNA as a predictive biomarker of HRD is described. This method detects both a global LOH signature used to evaluate genomic instability as well as gene-specific LOH in key HRR genes, and can be used to estimate the fraction of ctDNA in cfDNA. This method is an amplicon-based next-generation sequencing (NGS) approach in which the panel design, capture methodology, and LOH assessment methods are also specifically optimised to address the issues associated with the use of cfDNA as an analyte.

To overcome the challenges posed by cfDNA as an analyte, two components of the method of the present disclosure are highlighted. First, the application of molecular barcodes in amplicon primer design greatly enhances the accuracy and reproducibility of DNA molecule enumeration. This is useful in cfDNA samples where ctDNA fractions are low, as DNA enumeration is required for the determination of allelic ratios, as well as copy number evaluation. The second relates to the choice of workflow parameters for maximising DNA recovery. This includes (A) using an amplicon-based NGS workflow due to reportedly superior sensitivity as compared to hybrid-capture methods, and (B) optimising amplicon sizes to 120-220 bp, in accordance to the length of cfDNA fragments.

To overcome the challenges of detecting HRD in cfDNA, two additional components of the present disclosure are highlighted. First, the panel design is highly optimised to incorporate capture of two types of information, gene-specific LOH and a global LOH signature, while minimising the sequencing read cost of the panel. Second, the analysis method for global LOH determination is adapted for targeted panel sequencing, by utilising LOH information on the chromosome arm level, compared to length-based methods that require broader genomic coverage.

The present disclosure demonstrates that these features enable the detection of both gene-specific and global signatures of genetic instability, such as LOH, and as low as 10% tumour fraction from just 2.5 ng DNA, using a targeted NGS approach.

The method of the present disclosure therefore has the following advantages:
1. The unique design of the primer pairs allows the simultaneous capturing of SNPs across target chromosome arms and target genes, thereby enabling the determination of one or more signatures of genetic instability simultaneously at chromosome-level, gene-level and global-level.
2. The method of the present disclosure may be performed with only a small amount liquid nucleic acid sample (such as cfDNA) and tissue sample (such as tissue DNA), which improves cost-effectiveness.
3. The unique distribution of SNPs across the target chromosome arms and/or genes allows an informed call (i.e., the outcome of whether the sample is positive or negative for one or more signatures of genetic instability) to be made from a targeted panel of as low as approximately 1000 SNPs. This is in contrast with conventional genome-wide SNP genotyping approaches which requires the capture of at least 10000 SNPs in order to make an informed call.

The advantages described above allow the method of the present disclosure to be used in various commercial applications, such as the detection of HRD and other DNA repair deficiency disorders using non-invasive plasma cfDNA as an analyte, and the detection and quantification of tumour fraction (ctDNA) in cfDNA. In addition, the method of the present disclosure can also be used in the prediction of poly (ADP-ribose) polymerase inhibitor therapy response and the monitoring of poly (ADP-ribose) polymerase inhibitor treatment response over time. The kit as disclosed herein can also be used for the detection of DNA repair deficiency disorder, such as HRD, in cfDNA to inform clinical decisions for multiple cancer types.

In summary, the present disclosure describes for the first time:
1. The application of molecular barcodes for highly accurate enumeration of allelic ratios and DNA copy numbers.
2. Specific panel design with placement inclusion of SNPs at uniform intervals to allow capture of both genome-wide and gene-specific signatures of genetic instability, including LOH.
3. The specific design of amplicons and workflow to maximise nucleic acid (such as DNA) capture and informative polymorphic sites, ensuring compatibility with cfDNA as an analyte.
4. The design of data analysis workflows compatible with targeted SNP sequencing to elucidate chromosome-level, gene-level and global level signatures of genetic instability, including chromosome-level LOH, gene-specific LOH as well as global-level LOH.
5. The compatibility of the method of the present disclosure not just with cfDNA, but also with tissue DNA.

### SEQUENCE LISTING

| SEQ | | |
|---|---|---|
| ID NO | Sequence name | Sequence |
| 1 | Barcode sequence | NNNNNNNNNN |
| 2 | Partial adapter- | ACACGACGCTCTTCCGATCT |
| | specific sequence | |
| 3 | Partial adapter- | GACGTGTGCTCTTCCGATC |
| | specific sequence | |
| 4 | Full adapter-specific | AATGATACGGCGACCACCGAGATCTACACCTA |
| | sequence | GCGCTACACTCTTTCCCTACACGACGCTCTTC |
| | | CGATCT |
| 5 | Full adapter-specific | CAAGCAGAAGACGGCATACGAGATAACCGCGG |
| | sequence | GTGACTGGAGTTCAGACGTGTGCTCTTCCGAT |
| | | CT |
| 6 | Universal indexed | AATGATACGGCGACCACCGAGATCTACACCTA |
| | forward primer | GCGCTACACTCTTTCCCTACACGACGCTCTTC |
| | | CGATC*T |
| 7 | Universal indexed | CAAGCAGAAGACGGCATACGAGATAACCGCGG |
| | reverse primer | GTGACTGGAGTTCAGACGTGTGCTCTTCCGAT |
| | | C*T |
| 8 | An example of a | ACACGACGCTCTTCCGATCTNNNNNNNNNNTC |
| | “primer” when the | TTCTGAGGACTCTAATTTCTTGG |
| | target sequence is | |
| | BRCA1_SNP37 (an | |
| | example of forward | |
| | target capture primer, | |
| | illustrated in FIG. 2A) | |
| 9 | An example of | GACGTGTGCTCTTCCGATCTNNNNNNNNNNGA |
| | subsequent primers | TACTAGTTTTGCTGAAAATGACA |
| | for the “completion of | |
| | amplicon” (an | |
| | example of a reverse | |
| | target capture primer, | |
| | illustrated also in | |
| | FIG. 2A) | |
| 10 | An example of | TCTTCTGAGGACTCTAATTTCTTGGcccctct |
| | expected amplicon | tcggtaaccctgagccaaatgtgtatgggtga |
| | (only target-specific | aagggctaggactcctgctaagctctcctttc |
| | region) | tggacgcttttgctaaaaacagcagaactttc |
| | | cttaaTGTCATTTTCAGCAAAACTAGTATC |
| 11 | An example of the | ACACGACGCTCTTCCGATCTNNNNNNNNNNTC |
| | product after amplicon | TTCTGAGGACTCTAATTTCTTGGcccctcttc |
| | completion, illustrated | ggtaaccctgagccaaatgtgtatgggtgaaa |
| | also in FIG. 2A (in | gggctaggactcctgctaagctctcctttctg |
| | two steps) (Only one | gacgcttttgctaaaaacagcagaactttcct |
| | strand of the double | taaTGTCATTTTCAGCAAAACTAGTATCNNNN |
| | stranded product is | NNNNNNAGATCGGAAGAGCACACGTC |
| | shown) | |
| 12 | An example of the | AATGATACGGCGACCACCGAGATCTACACCTA |
| | final product, | GCGCTACACTCTTTCCCTACACGACGCTCTTC |
| | illustrated also in | CGATCTNNNNNNNNNNTCTTCTGAGGACTCTA |
| | FIG. 2A (suitable | ATTTCTTGGcccctcttcggtaaccctgagcc |
| | for sequencing on | aaatgtgtatgggtgaaagggctaggactcct |
| | Illumina) | gctaagctctcctttctggacgcttttgctaa |
| | | aaacagcagaactttccttaaTGTCATTTTCA |
| | | GCAAAACTAGTATCNNNNNNNNNNAGATCGGA |
| | | AGAGCACACGTCTGAACTCCAGTCACCCGCGG |
| | | TTATCTCGTATGCCGTCTTCTGCTTG |

1. A method of detecting the presence or absence of one or more signatures of genetic instability at chromosome-level and/or gene-level within a nucleic acid sample, comprising the steps of:
(a) identifying a plurality of single nucleotide polymorphism (SNPs) at one or more pre-determined intervals across:
(I) one or more target chromosome arms, wherein each target chromosome arm comprises a plurality of genes; and/or
(II) one or more target genes;
(b) performing a plurality of multiplexed PCR reactions using:
(I) a plurality of forward and reverse primer pairs that are capable of capturing the plurality of SNPs identified across the one or more target chromosome arms in step (a)(I),
wherein each primer of the plurality of forward and reverse primer pairs comprises a target-specific sequence capable of capturing at least one SNP in the plurality of the SNPs identified across the one or more target chromosome arms in step (a)(I),
wherein each forward primer and/or reverse primer of the plurality of forward and reverse primer pairs comprise(s) a barcode sequence on the 5′ end of the target-specific sequence,
wherein each primer of the plurality of forward and reverse primer pairs comprises an adapter-specific sequence; and/or
(II) a plurality of forward and reverse primer pairs that are capable of capturing the plurality of SNPs identified across the one or more target genes in step (a)(II),
wherein each primer of the plurality of forward and reverse primer pairs comprises a target-specific sequence capable of capturing at least one SNP in the plurality of the SNPs identified across the one or more target genes in step (a)(II),
wherein each forward primer and/or reverse primer of the plurality of forward and reverse primer pairs comprise(s) a barcode sequence on the 5′ end of the target-specific sequence,
wherein each primer of the plurality of forward and reverse primer pairs comprises an adapter-specific sequence,
thereby generating a plurality of amplicons;
(c) using the plurality of amplicons from step (b) to generate a plurality of sequencing reads with a next-generation sequencing platform;
(d) deriving a consensus sequence read of each sequence from the plurality of sequencing reads obtained from step (c);
(e) performing a sequence alignment of the consensus sequence reads obtained from step (d) to a reference genome;
(f) performing variant calling based on the sequence alignment obtained from step (e) to calculate variant allele frequency (VAF);
(g) determining and enumerating a plurality of informative polymorphic sites from the VAF obtained in step (f), wherein an informative polymorphic site is defined as a site comprising between 5% and 95% VAF;
(h) calculating the allelic ratio (AR) at each informative polymorphic site of the plurality of informative polymorphic sites determined in step (g), wherein AR is defined as a ratio of a major allele A to a minor allele B, wherein
(I) if the AR at an informative polymorphic site is equal to or higher than a pre-determined threshold value, said informative polymorphic site is classified as “genetically unstable”; and
(II) if the AR at an informative polymorphic site is lower than a pre-determined threshold value, said informative polymorphic site is classified as “genetically stable” (not genetically unstable); and
(i) determining whether the one or more target chromosome arms and/or the one or more target genes are “positive” for one or more signatures of genetic instability, wherein
(I) if a target chromosome arm comprises a minimum pre-determined number of informative polymorphic sites obtained from step (g) and if at least 50% of the informative polymorphic sites are classified as “genetically unstable” in step (h)(I), said target chromosome arm is determined to be “positive” for one or more signatures of genetic instability at chromosome-level; and/or
(II) if a target gene comprises a minimum pre-determined number of informative polymorphic sites obtained from step (g) and if at least 30% of the informative polymorphic sites are classified as “genetically unstable” in step (h)(I), said target gene is determined to be “positive” for one or more signatures of genetic instability at gene-level;
wherein if the one or more target chromosome arms and/or the one or more target genes are determined to be “positive”, then one or more signatures of genomic instability are determined to be present at chromosome-level and/or gene-level within the nucleic acid sample, and
wherein if there is/are no target chromosome arm and/or target gene that is/are determined to be “positive”, then one or more signatures of genomic instability are determined to be absent at chromosome-level and/or gene-level within the nucleic acid sample;
thereby detecting the presence or absence of one or more signatures of genomic instability at chromosome-level and/or gene-level within the nucleic acid sample based on the results obtained in step (i).

2. The method of claim 1, wherein the minimum pre-determined number of informative polymorphic sites in step (i)(I) is 4 and/or the minimum pre-determined number of informative polymorphic sites in step (i)(II) is 3.

3. The method of claim 1, wherein the one or more signatures of genetic instability are selected from the group consisting of loss of heterozygosity (LOH), large-scale state transitions (LST), and telomeric allelic imbalance (TAI).

4. The method of claim 3, wherein the one or more signatures of genetic instability are LOH and/or TAI, the method further comprises determining whether the LOH and/or TAI are associated with allelic copy number alteration by:
(j) enumerating the number of allelic copies at the plurality of informative polymorphic sites, wherein if the plurality of informative polymorphic sites are classified as “genetically unstable” in step (h)(I) and
(I) if there is a decrease (loss) in the number of allelic copies, the one or more signatures of genetic instability are determined to be “copy-number-loss signature”;
(II) if there is an increase (gain) in the number of allelic copies, the one or more signatures of genetic instability are determined to be “copy-number-gain signature”; and
(III) if there is no change in the number of allelic copies, the one or more signatures of genetic instability are determined to be “copy-neutral signature”.

5. The method of claim 3, wherein the signature of genetic instability is LOH.

6. The method of claim 1, wherein the nucleic acid sample is selected from the group consisting of DNA sample and RNA sample, wherein optionally the nucleic acid sample is a DNA sample, wherein optionally the DNA sample is cell-free DNA (cfDNA) or DNA encapsulated within tissues and/or cells, and wherein optionally the DNA sample is cfDNA.

7. The method of claim 1, wherein the nucleic acid sample is selected from the group consisting of a liquid sample, a tissue sample, and a cell sample.

8. The method of claim 7, wherein the liquid sample is a bodily fluid, wherein optionally the bodily fluid is selected from the group consisting of blood, bone marrow, cerebral spinal fluid, peritoneal fluid, pleural fluid, lymph fluid, ascites, serous fluid, sputum, lacrimal fluid, stool, urine, saliva, ovarian fluid, oviductal fluid, prostatic fluid, ductal fluid from breast, gastric juice and pancreatic juice, wherein optionally the bodily fluid is blood, and wherein optionally the blood is plasma.

9. The method of claim 7, wherein the tissue sample is a frozen tissue sample or a fixed tissue sample, and wherein optionally the fixed tissue sample is a Formalin-Fixed Paraffin-Embedded (FFPE) tissue sample.

10. The method of claim 1, wherein the one or more target chromosome arms are selected from any chromosomes found in a subject, wherein optionally the chromosomes of the subject comprise autosomal chromosomes.

11. The method of claim 1, wherein the method further comprises determining the presence or absence of one or more signatures of genetic instability at global-level within the nucleic acid sample by:
(k) enumerating the number of target chromosome arms and/or target genes determined to be “positive” for one or more signatures of genetic instability at chromosome-level and/or gene-level in step (i); and
(l) calculating the percentage of the total number of target chromosome arms and/or target genes determined to be “positive” for one or more signatures of genetic instability obtained from step (k) divided by the total number of target chromosome arms and/or target genes in step (a).

12. The method of claim 1, wherein the one or more target genes are selected from the group consisting of AT-rich interaction domain 1A (ARIDIA), ATM serine/threonine kinase (ATM), ATR serine/threonine kinase (ATR), ATRX chromatin remodeler (ATRX), BRCA1 associated protein 1 (BAP1), BRCA1 associated RING domain 1 (BARD1), BLM RecQ like helicase (BLM), BRCA1 DNA repair associated (BRCA1), BRCA2 DNA repair associated (BRCA2), BRCA1 interacting helicase 1 (BRIP1), cyclin dependent kinase 12 (CDK12), Checkpoint kinase 1 (CHEK1), Checkpoint kinase 2 (CHEK2), EMSY transcriptional repressor, BRCA2 interacting (EMSY), FA complementation group A (FANCA), FA complementation group C (FANCC), FA complementation group D2 (FANCD2), FA complementation group E (FANCE), FA complementation group F (FANCF), FA complementation group G (FANCG), FA complementation group I (FANCI), FA complementation group L (FANCL), FA complementation group M (FANCM), MRE11 homolog, double strand break repair nuclease (MRE11), nibrin (NBN), Partner and localizer of BRCA2 (PALB2), Phosphatase and tensin homolog (PTEN), RAD50 double strand break repair protein (RAD50), RAD51 recombinase (RAD51), RAD51 paralog B (RAD51B), RAD51 paralog C (RAD51C), RAD51 paralog D (RAD51D), RAD52 homolog, DNA repair protein (RAD52), RAD54 like (RAD54L), Replication protein A1 (RPA1), and X-ray repair cross complementing 2 (XRCC2).

13. The method of claim 1, wherein:
(A) the one or more pre-determined intervals for the plurality of SNPs identified across the one or more target chromosome arms in step (a)(I) comprise 1 to 20 megabases (Mb); and/or
(B) the one or more pre-determined intervals for the plurality of SNPs identified across the one or more target genes in step (a)(II) comprise 2 to 300 kilobases (kb).

14. The method of claim 1, wherein the barcode sequence is an oligonucleotide comprising 10 to 16 random nucleotides, wherein optionally the barcode sequence is an oligonucleotide comprising 10 random nucleotides.

15. The method of claim 1, wherein the length of the plurality of amplicons generated in step (b) is 100 to 250 base pairs.

16. The method of claim 1, wherein the nucleic acid sample is obtained from a subject having and/or suspected of having a disorder associated with one or more signatures of genetic instability.

17. The method of claim 16, wherein the disorder is a DNA repair deficiency disorder, wherein the DNA repair deficiency disorder is selected from the group consisting of Homologous Recombination Deficiency (HRD), Non-Homologous End-Joining (NHEJ) Deficiency, DNA mismatch repair (MMR) deficiency, nucleotide excision repair (NER) deficiency, and base excision repair (BER) deficiency, wherein optionally the DNA repair deficiency disorder is HRD.

18. The method of claim 17, wherein the subject has or is suspected of having a DNA repair deficiency disorder, if one or more signatures of genetic instability are present at gene-level, chromosome-level and/or global-level within the nucleic acid sample.

19. The method of claim 17, wherein the DNA repair deficiency disorder is associated with cancer, wherein optionally the cancer is selected from the group consisting of ovarian cancer, prostate cancer, breast cancer, leukaemia, lung cancer, colorectal cancer, pancreatic cancer, nasopharyngeal cancer, liver cancer, cholangiocarcinoma, oesophageal cancer, urothelial cancer, and gastrointestinal cancer, endometrial cancer, peritoneal cancer, cervical cancer, thyroid cancer, kidney cancer, and brain cancer.

20. The method of claim 19, wherein the nucleic acid sample is cfDNA, and wherein the method further comprises using the AR ratio obtained from step (h) to determine the fraction of tumour-derived circulating DNA (ctDNA) that may be present within the cfDNA sample.

21. A kit for detecting the presence or absence of one or more signatures of genetic instability at chromosome-level and/or gene-level within a nucleic acid sample according to the method of claim 1, wherein the kit comprises:
a plurality of forward and reverse primer pairs that are capable of capturing a plurality of SNPs identified across one or more target chromosome arms; and/or
a plurality of forward and reverse primer pairs that are capable of capturing a plurality of SNPs identified across one or more target genes.

22. The kit of claim 21, wherein the kit further comprises:
a buffer for performing a plurality of multiplexed PCR reactions;
universal indexed adapter primers;
a DNA polymerase; and
a plurality of deoxynucleoside triphosphates (dNTPs),
wherein optionally the kit further comprises an exonuclease.

23. A method of predicting and/or monitoring the response of a subject having a disorder associated with one or more signatures of genetic instability towards treatment with one or more poly (ADP-ribose) polymerase inhibitors, comprising detecting the presence or absence of one or more signatures of genetic instability at chromosome-level and/or gene level according to the method of claim 1.

24. The method of claim 23, wherein the disorder is a DNA repair deficiency disorder, wherein the DNA repair deficiency disorder is selected from the group consisting of Homologous Recombination Deficiency (HRD), Non-Homologous End-Joining (NHEJ) Deficiency, DNA mismatch repair (MMR) deficiency, nucleotide excision repair (NER) deficiency, and base excision repair (BER) deficiency, wherein optionally the DNA repair deficiency disorder is HRD.


SEQUENCE LISTING (12 sequences)

SEQ ID NO: 1 - skipped

SEQ ID NO: 2 - DNA, 20 bp, synthetic construct
  source 1..20: mol_type = other DNA
acacgacgctcttccgatct

SEQ ID NO: 3 - DNA, 19 bp, synthetic construct
  source 1..19: mol_type = other DNA
gacgtgtgctcttccgatc

SEQ ID NO: 4 - DNA, 70 bp, synthetic construct
  source 1..70: mol_type = other DNA
aatgatacggcgaccaccgagatctacacctagcgctacactctttccctacacgacgctcttccgatct

SEQ ID NO: 5 - DNA, 66 bp, synthetic construct
  source 1..66: mol_type = other DNA
caagcagaagacggcatacgagataaccgcgggtgactggagttcagacgtgtgctcttccgatct

SEQ ID NO: 6 - DNA, 70 bp, synthetic construct; identical to SEQ ID NO: 4
  source 1..70: mol_type = other DNA

SEQ ID NO: 7 - DNA, 66 bp, synthetic construct; identical to SEQ ID NO: 5
  source 1..66: mol_type = other DNA

SEQ ID NO: 8 - DNA, 55 bp, synthetic construct
  source 1..55: mol_type = other DNA
acacgacgctcttccgatctnnnnnnnnnntcttctgaggactctaatttcttgg

SEQ ID NO: 9 - DNA, 55 bp, synthetic construct
  source 1..55: mol_type = other DNA
gacgtgtgctcttccgatctnnnnnnnnnngatactagttttgctgaaaatgaca

SEQ ID NO: 10 - DNA, 158 bp, synthetic construct
  source 1..158: mol_type = other DNA
tcttctgaggactctaatttcttggcccctcttcggtaaccctgagccaaatgtgtatgggtgaaagggctaggactcctgctaagctctcctttctggacgcttttgctaaaaacagcagaactttccttaatgtcattttcagcaaaactagtatc

SEQ ID NO: 11 - DNA, 218 bp, synthetic construct
  source 1..218: mol_type = other DNA
acacgacgctcttccgatctnnnnnnnnnntcttctgaggactctaatttcttggcccctcttcggtaaccctgagccaaatgtgtatgggtgaaagggctaggactcctgctaagctctcctttctggacgcttttgctaaaaacagcagaactttccttaatgtcattttcagcaaaactagtatcnnnnnnnnnnagatcggaagagcacacgtc

SEQ ID NO: 12 - DNA, 314 bp, synthetic construct
  source 1..314: mol_type = other DNA
aatgatacggcgaccaccgagatctacacctagcgctacactctttccctacacgacgctcttccgatctnnnnnnnnnntcttctgaggactctaatttcttggcccctcttcggtaaccctgagccaaatgtgtatgggtgaaagggctaggactcctgctaagctctcctttctggacgcttttgctaaaaacagcagaactttccttaatgtcattttcagcaaaactagtatcnnnnnnnnnnagatcggaagagcacacgtctgaactccagtcacccgcggttatctcgtatgccgtcttctgcttg